(19) United States
(12) Patent Application Publication
(10) Pub. No.: US 2008/0242709 A1
(43) Pub. Date: Oct. 2, 2008

(54) 2-AMIDO-4-ISOXAZOLYL THIAZOLE COMPOUNDS EXHIBITING ATP-UTILIZING ENZYME INHIBITORY ACTIVITY, AND COMPOSITIONS, AND USES THEREOF

(76) Inventors: John K. Dickson, Apex, NC (US); Carl Nicholas Hodge, Los Gatos, CA (US); Ke Chen, Cary, NC (US)

Correspondence Address: GENENTECH, INC., 1 DNA WAY, SOUTH SAN FRANCISCO, CA, 94080, US

(21) Appl. No.: 11/836,528

(22) Filed: Aug. 9, 2007

Related U.S. Application Data
(60) Provisional application No. 60/838,243, filed on Aug. 16, 2006.

(51) Int. Cl. A61K 31/427; C07D 277/28; A61P 35/00

(52) U.S. Cl. 514/371; 548/195

(57) ABSTRACT

2-Amido-4-isoxazolyl thiazole compounds exhibiting ATP-utilizing enzyme inhibitory activity, methods of using compounds exhibiting ATP-utilizing enzyme inhibitory activity, and compositions comprising compounds exhibiting ATP-utilizing enzyme inhibitory activity, are disclosed.

This non-provisional application claims the benefit of priority of U.S. Provisional Application Ser. No. 60/838,243 filed on 16 Aug. 2006, which is incorporated by reference in its entirety.

### FIELD OF THE INVENTION

The invention relates generally to compounds with anti-cancer activity and more specifically to compounds which inhibit protein kinase activity, including AKT and PIM. The invention also relates to methods of using the compounds for in vitro, in situ, and in vivo diagnosis or treatment of mammalian cells, or associated pathological conditions

### BACKGROUND OF THE INVENTION

ATP-utilizing enzymes catalyze the transfer of a phosphate group from an adenosine triphosphate (ATP) molecule to a biomolecule such as a protein or carbohydrate. Examples of ATP-utilizing enzymes include, but are not limited to, synthetases, ligases, and kinases.

Protein kinases encompass a large family of functionally and structurally related enzymes that are responsible for the control of a wide variety of cellular processes including signal transduction, metabolism, transcription, cell cycle progression, cytoskeletal rearrangement and cell movement, apoptosis, and differentiation. In general, protein kinases control protein activity by catalyzing the addition of a negatively charged phosphate group from a phosphate-containing molecule such as cyclic adenosine monophosphate (cAMP), adenosine diphosphate (ADP), and ATP, to other proteins. Protein phosphorylation in turn can modulate or regulate the functioning of a target protein. Protein phosphorylation is known to play a role in intercellular communication during development, in physiological responses and in homeostasis, and in the functioning of the nervous and immune systems.

The unregulated phosphorylation of proteins is known to be a cause of, or associated with the etiology of major diseases, such as Alzheimer's disease, stroke, diabetes,

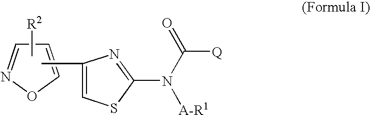

and pharmaceutically acceptable salts, solvates, chelates, non-covalent complexes, prodrugs, and mixtures thereof, wherein

R¹ is a 5- to 7-membered cycloheteroalkyl ring which optionally includes 1 or 2 additional heteroatoms chosen from O, S, and N in the ring and where the ring is further substituted with a group R³;

R² is chosen from phenyl and substituted phenyl;

Q is chosen from thienyl and substituted thienyl;

A is chosen from 1,3-propylene and 1,4-butylene; and

R³ is —C(O)NR⁴R⁵ wherein R⁴ and R⁵ are independently chosen from hydrogen, hydroxy, hydroxyethyl, lower alkyl, and lower alkoxy.

Formula I compounds include the structures:

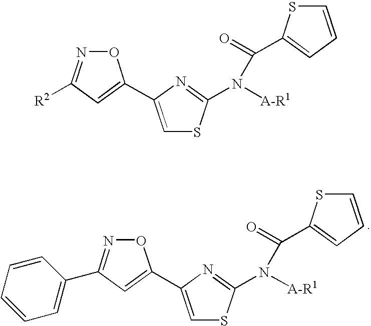

In certain embodiments, R¹ is chosen from pyrrolidine, piperidine, azepane, piperazine, and morpholine, each of which is further substituted with a group R³.

In certain embodiments, R¹ is chosen from piperidine further substituted with a group R³.

In certain embodiments, R⁴ is hydrogen.

In certain embodiments, R⁵ is chosen from hydrogen, hydroxy, hydroxyethyl, and lower alkyl.

In certain embodiments, R⁵ is chosen from hydrogen, hydroxy, hydroxyethyl, and methyl.

In certain embodiments, R² is phenyl.

In certain embodiments, Q is thienyl.

In certain embodiments, A is 1,3-propylene. obesity, inflammation, cancer, and rheumatoid arthritis. Deregulated protein kinase activity and over-expression of protein kinases has been implicated in the pathophysiology of a number of important human disorders. Furthermore, genetic mutations in protein kinases are implicated in a number of disorders and many toxins and pathogens exert their effects by altering the phosphorylation of intracellular proteins.

ATP-utilizing enzymes, such as protein kinases, therefore, represent a broad class of pharmacological targets of interest for the treatment of human disease. The identification and development of compounds that selectively inhibit the functioning of ATP-utilizing enzymes is therefore of considerable interest.

AKT/protein kinase B (PKB) is a pivotal kinase in the phosphatidylinositol 3′-OH kinase (PI3K)/AKT pathway that regulates cell survival and apoptosis, or programmed cell death (Kauffmann-Zeh et al., Nature 385:544-548 (1997); Hemmings, Science, 275:628-630 (1997); Dudek et al., Science 275:661-665 (1997)). The PI3K/AKT pathway is activated by numerous factors, including growth factors such as platelet-derived growth factor and insulin-like growth factor-1, and this activation involves the induction of PI3K activity to increase the levels of its product phosphatidylinositol (3,4,5)-triphosphate (PIP3) and the subsequent recruitment of AKT to the PIP3-enriched membrane via its plekstrin homology (PH) domain (Hemmings Science, 277:534 (1997). AKT is subsequently activated via phosphorylation, and the two regulatory sites are Thr308 and Ser473. The tumor suppressor PTEN is a protein and lipid phosphatase that negatively regulates the PI3K/AKT pathway by removing the 3′ phosphate of PIP3. There are three isoforms of AKT: AKT1 (PKB alpha), AKT2 (PKB beta) and AKT3(PKB gamma).

Numerous lines of evidence has linked the PI3K/AKT pathway to human diseases, particularly cancer (Vivanco and Sawyers, Nature Rev. Cancer 2:489-501 (2002); Luo et al., Cancer Cell 4:257-262 (2003); Vivanco and Sawyer, 2002 Nature Rev. Drug Disc. 2, 489-501; Bellacosa et al., Canc. Biol. Therapy, 3, 268-275 (2004)). AKTs are differentially overexpressed in a number of human tumors (Sun et al., Am. J. Pathol. 159:431-437 (2001); Yuan et al., Oncogene 19:2324-2330 (2000); Nakatani et al., J. Biol. Chem. 274:21528-21532 (1999)) and AKT1 and AKT2 have been shown to be amplified in several cancer types (Staal, Proc. Natl. Acad. Sci. USA 84:5034-5037 (1987); Nicholsen and Anderson, Cell. Signaling 14,381-395 (2002)). Further, AKT activation in human cancers has been demonstrated to occur by other means, including mutation of the tumor suppressor PTEN (Di Cristofano and Pandolfi, Cell 100:387-390 (2000); Sun et al., Proc. Natl. Acad. Sci. USA 96:6199-6204 (1999)). One consequence of PTEN loss is hyperactivation of AKT and phosphorylation of downstream AKT substrates, including BAD, FOXO proteins and GSK3. Deletion of AKT1 has been shown to reverse the aggressive growth phenotype of PTEN null mouse embryonic stem cells (Stiles et al., Mol. Cell. Biol. 22:3842-3851 (2002) Loss-of function mutations in the PTEN gene are extremely common among sporadic glioblastomas, melanoma, prostate cancers and endometrial carcinomas, and a significant percentage of breast tumors, lung cancers, and lymphomas have PTEN mutations (Cantley and Neel, (1999) Proc. Natl. Acad. Sci. USA 96:4240-4245; Luo et al. (2003) Cancer Cell, 4:257-262). Mutations of PIK3CA which encodes p110α catalytic subunit of class 1A PI3Ks results in activating mutations of PI3K (Samuels et al., Cancer Cell 7:561-573 (2005)). PIK3CA appears to be one of the most highly mutated oncogenes, with somatic mutations seen in colorectal, gastric, breast, and certain brain tumors (Samuels et al., Cancer Cell 7,561-573 (2005) and references therein). Together, these data indicate that AKTs play a key role in tumor biology and that the three AKT isoforms may serve different functions; therefore, selective inhibition of one or more AKT isoenzyme may be a productive approach to cancer therapeutics.

Blocking of the PI3K/AKT pathway could inhibit the proliferation of tumor cells and sensitize them toward apoptosis. The resistance of many types of cancer to conventional chemotherapies is a major factor undermining successful cancer treatment, and targeting the PI3K/AKT pathway for inhibition is being investigated as a strategy to overcome chemotherapeutic resistance (McCormick, Nature, 428, 267-269 (2004); Bellacosa et al., Canc. Biol. Therapy, 3, 268-275 (2004); West et al., Drug Resistance Update 5, 234-248 (2002); Bianco et al., Oncogene 22, 2812-2822 (2003)). Therefore, conventional targeted and cytotoxic anti-proliferative and targeted anti-angiogenic therapeutics would complement the pro-apoptotic mechanism of an AKT inhibitor.

A number of cancers are associated with activation of the P13K/AKT pathway, including, but not limited to, glioblastoma, ovarian, breast, endometrial, hepatocellular carcinoma, melanoma, digestive tract, lung, renal-cell carcinoma, thyroid, lymphoid, prostate and pancreatic cancer (Vivanco and Sawyer, Nature Rev. Drug Disc., 2, 489-501 (2002); Graff, Expert Opin. Ther. Targets, 6, 103-113 (2002); Bondar et al., Mol. Canc. Therapies 1, 989-997 (2002)).

Inappropriate activation of the phosphatidylinositol 3-kinase (PI3K)/AKT pathway has also been associated with the development of diseases such as diabetes and autoimmunity.

The PI3K/AKT pathway also functions in the growth and survival of normal tissues and may be regulated during normal physiology to control cell and tissue function. Thus the undesirable proliferation and survival of normal cells and tissues may result in a number of disorders, such as disorders of immune cells associated with prolonged expansion and survival of cell population leading to prolonged or up regulated immune response. For example, T and B lymphocytes response to cognate antigens or growth factors such as Il-2 activates the PI3K/AKT pathway and is responsible for maintaining the survival of the antigen specific lymphocyte clones during the immune response. Under conditions in which lymphocytes and other immune cells are responding to inappropriate self or foreign antigens, or in which other abnormalities lead to prolonged activation, the PI3K/AKT pathway contributes an important survival signal preventing normal mechanism by which the immune response is terminated via apoptosis of the activated cell population. There is a considerable amount of evidence demonstrating the expansion of lymphocyte populations responding to self antigens in autoimmune conditions such as multiple sclerosis and arthritis. Expansion of lymphocyte populations responding to inappropriately to foreign antigens is a feature of another set of conditions such as allergic response and asthma.

Other examples of inappropriate expansion, growth, proliferation, hyperplasia and survival of normal cells in which the PI3K/AKT pathway may play a role include, but are not limited to, atherosclerosis, cardiac myopathy and glomerulonephritis.

In addition to the role in cell growth and survival, the PI3K/AKT pathway functions in the control of glucose metabolism by insulin. As a consequence, modulators of PI3K/AKT activity may also find utility in disease in which there is a dysfunction of glucose metabolism and energy storage such as diabetes, metabolic disease and obesity.

AKT was first identified as a viral oncogene (Bellacosa et al. 1991 Science 254:274-277). A number of studies have demonstrated the role of PI3K/AKT pathway in the life cycle of numerous viruses. Some viral proteins have been shown to activate the PI3K/AKT pathway, thus providing an environment favorable for viral replication. These include the Tat protein of HIV (Borgatti et al. 1997, Eur. J. Immunol. 27:2805-2811), protein X of hepatitis B virus (Lee et al. 2001 J. Biol. Chem. 276:16969-16977), NS5A of hepatitis C virus (He et al. 2002 J. Virol. 76:9207-9217), human cytomegalovirus (Johnson et al. 2001 J. Virol. 75:6022-6032), and Epstein-Barr virus (Moody et al. 2005 J. Virol. 79:5499-5506).

ATP-utilizing enzymes, such as protein kinases, therefore, represent a broad class of pharmacological targets of interest for the treatment of human disease. The identification and development of compounds that selectively inhibit the functioning of ATP-utilizing enzymes is therefore of considerable interest.

2-Amido-thiazole compounds are described in US 2006/0052416 and demonstrated to have ATP-utilizing enzyme inhibitory activity, including AKT1 binding activity.

### SUMMARY OF THE INVENTION

Provided is at least one chemical entity chosen from compounds of Formula I:

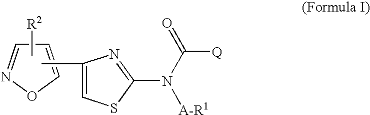

and pharmaceutically acceptable salts, chelates, non-covalent complexes, and mixtures thereof, wherein

R¹ is a 5- to 7-membered cycloheteroalkyl ring which optionally includes 1 or 2 additional heteroatoms chosen from O, S, and N in the ring and where the ring is further substituted with a group R³;

R² is chosen from phenyl and substituted phenyl;

Q is chosen from thienyl and substituted thienyl;

A is chosen from 1,3-propylene and 1,4-butylene; and

R³ is —C(O)NR⁴R⁵ wherein R⁴ and R⁵ are independently chosen from hydrogen, hydroxy, hydroxyethyl, lower alkyl, and lower alkoxy.

Also provided is a pharmaceutical composition comprising at least one pharmaceutically acceptable vehicle, and a therapeutically effective amount of at least one chemical entity of the present disclosure.

Also provided is a packaged pharmaceutical formulation comprising a pharmaceutical composition comprising at least one pharmaceutically acceptable vehicle and a therapeutically effective amount of at least one chemical entity of the present disclosure; and instructions for using the composition to treat a mammal.

Also provided is a method of treating at least one disease in a patient in need of such treatment comprising administering to the patient a therapeutically effective amount of at least one chemical entity of the present disclosure.

Additional embodiments of the invention are set forth in the description which follows, or may be learned by practice of the invention.

### DETAILED DESCRIPTION OF EXEMPLARY EMBODIMENTS

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature, patents, and similar materials differs from or contradicts this application, including but not limited to defined terms, term usage, described techniques, or the like, this application controls.

### DEFINITIONS

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements. At the very least, and not as an attempt to limit the application of the doctrine of equivalents to the scope of the claims, each numerical parameter as set forth in the claims should at least be construed in light of the number of reported significant digits and by applying ordinary rounding techniques.

Unless stated otherwise, the following terms and phrases as used herein are intended to have the following meanings:

“Acyl” refers to a radical —C(O)R, where R is hydrogen, alkyl, substituted alkyl, substituted cycloalkyl, substituted heterocycloalkyl, substituted aryl, or substituted heteroaryl group as defined herein. Representative examples include, but are not limited to, formyl, acetyl, cyclohexylcarbonyl, cyclohexylmethylcarbonyl, benzoyl, benzylcarbonyl, and the like.

“Alkanyl” refers to a saturated branched, straight-chain or cyclic alkyl group derived by the removal of one hydrogen atom from a single carbon atom of a parent alkane. Typical alkanyl groups include, but are not limited to, methanyl; ethanyl; propanyls such as propan-1-yl, propan-2-yl(isopropyl), cyclopropan-1-yl; butanyls such as butan-1-yl, butan-2-yl (sec-butyl), 2-methyl-propan-1-yl(isobutyl), 2-methyl-propan-2-yl(t-butyl), cyclobutan-1-yl; and the like.

“Alkenyl” refers to an unsaturated branched, straight-chain or cyclic alkyl group having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl; and the like. In certain embodiments, an alkenyl group has from 2 to 20 carbon atoms and in other embodiments, from 2 to 6 carbon atoms.

“Alkoxy” refers to a radical —OR where R represents an alkyl, substituted alkyl, substituted cycloalkyl, substituted heterocycloalkyl, substituted aryl, or substituted heteroaryl group as defined herein. Representative examples include, but are not limited to, methoxy, ethoxy, propoxy, butoxy, cyclohexyloxy, and the like.

“Alkoxycarbonyl” refers to a radical —C(O)— alkoxy where alkoxy is as defined herein.

“Alkyl” refers to a saturated or unsaturated, branched, straight-chain or cyclic monovalent hydrocarbon group derived by the removal of one hydrogen atom from a single carbon atom of a parent alkane, alkene or alkyne. Typical alkyl groups include, but are not limited to, methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl(allyl), cycloprop-1-en-1-yl; cycloprop-2-en-1-yl, prop-1-yn-1-yl, prop-2-yn-1-yl; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl, but-2-en-2-yl, buta-1, 3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Where a specific level of saturation is intended, the expressions “alkanyl,” “alkenyl,” and “alkynyl” are used. In certain embodiments, an alkyl group comprises from 1 to 20 carbon atoms. In other embodiments, an alkyl group comprises from 1 to 6 carbon atoms, and is referred to as a lower alkyl group.

The term “substituted amino” refers to the group —NHR^{d} or —NR^{d}R^{d} where each R^{d} is independently chosen from: alkyl, substituted alkyl, cycloalkyl, substituted cycloalkyl, acyl, substituted acyl, aryl, substituted aryl, heteroaryl, substituted heteroaryl, heterocycloalkyl, substituted heterocycloalkyl, alkoxycarbonyl, and sulfonyl. Representative examples include, but are not limited to, dimethylamino, methylethylamino, di-(1-methylethyl)amino, (cyclohexyl)(methyl)amino, (cyclohexyl)(ethyl)amino, (cyclohexyl)(propyl)amino, and the like.

“Sulfonyl” refers to a radical —S(O)₂R where R is an alkyl, substituted alkyl, substituted cycloalkyl, substituted heterocycloalkyl, substituted aryl, or substituted heteroaryl group as defined herein. Representative examples include, but are not limited to methylsulfonyl, ethylsulfonyl, propylsulfonyl, butylsulfonyl, and the like.

“Sulfinyl” refers to a radical —S(O)R where R is an alkyl, substituted alkyl, substituted cycloalkyl, substituted heterocycloalkyl, substituted aryl, or substituted heteroaryl group as defined herein. Representative examples include, but are not limited to, methylsulfinyl, ethylsulfinyl, propylsulfinyl, butylsulfinyl, and the like.

“Sulfanyl” refers to a radical —SR where R is an alkyl, substituted alkyl, substituted cycloalkyl, substituted heterocycloalkyl, substituted aryl, or substituted heteroaryl group as defined herein. Representative examples include, but are not limited to, methylthio, ethylthio, propylthio, butylthio, and the like.

“Alkynyl” refers to an unsaturated branched, straight-chain or cyclic alkyl group having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl; butynyls such as but-1-yn-1-yl, but-1-yn-3-yl, but-3-yn-1-yl; and the like. In certain embodiments, an alkynyl group has from 2 to 20 carbon atoms and in other embodiments, from 3 to 6 carbon atoms.

“Amino” refers to the radical —NH₂.

“Aminocarbonyl” refers to the group —C(O)NRR′ where R and R′ are independently chosen from hydrogen, alkyl, substituted alkyl, substituted cycloalkyl, substituted heterocycloalkyl, substituted aryl, or substituted heteroaryl group as defined herein, or optionally R′ and R″ together with the nitrogen atom to which R and R′ are attached form one or more heterocyclic or substituted heterocyclic rings.

“Aryl” encompasses: 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. For example, aryl includes 5- and 6-membered carbocyclic aromatic rings fused to a 5- to 7-membered heterocycloalkyl ring containing 1 or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined herein.

“Arylalkyl” or “aralkyl” refers to an acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, and/or arylalkynyl is used. In certain embodiments, an arylalkyl group can be (C₆₋₃₀) arylalkyl, e.g., the alkanyl, alkenyl or alkynyl moiety of the arylalkyl group can be (C₁₋₁₀) and the aryl moiety can be (C₆₋₂₀).

“Aryloxycarbonyl” refers to a radical —C(O)—O—R wherein R is chosen from aryl and substituted aryl as defined herein.

“Carbonyl” refers to the radical —C(O).

“Carboxy” refers to the radical —C(O)OH.

“Cleave” refers to breakage of chemical bonds and is not limited to chemical or enzymatic reactions or mechanisms unless clearly indicated by the context.

When the chemical structure and chemical name conflict, the chemical structure is determinative of the identity of the compound. The chemical entities of the present disclosure may contain one or more chiral centers and/or double bonds and therefore, may exist as stereoisomers, such as double-bond isomers (i.e., geometric isomers), enantiomers or diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into the component enantiomers or stereoisomers using separation techniques or chiral synthesis techniques well known to the skilled artisan.

Compounds of Formula I include, but are not limited to, optical isomers of compounds of Formula I, racemates, and other mixtures thereof. In those situations, the single enantiomers or diastereomers, i.e., optically active forms, can be obtained by asymmetric synthesis or by resolution of the racemates. Resolution of the racemates can be accomplished, for example, by conventional methods such as crystallization in the presence of a resolving agent, or chromatography, using, for example a chiral high-pressure liquid chromatography (HPLC) column. In addition, compounds of Formula I include Z- and E-forms (or cis- and trans-forms) of compounds with double bonds. Where compounds of Formula I exists in various tautomeric forms, chemical entities of the present disclosure include all tautomeric forms of the compound.

Chemical entities of the present disclosure include, but are not limited to, compounds of Formula 1 and all pharmaceutically acceptable forms thereof. Pharmaceutically acceptable forms of the compounds recited herein include pharmaceutically acceptable salts, solvates, crystal forms (including polymorphs and clathrates), chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, solvates, chelates, non-covalent complexes, prodrugs, and mixtures.

The term “chelate” refers to the chemical entity formed by the coordination of a compound to a metal ion at two (or more) points.

The term “non-covalent complex” refers to the chemical entity formed by the interaction of a compound and another molecule wherein a covalent bond is not formed between the compound and the molecule. For example, complexation can occur through van der Waals interactions, hydrogen bonding, and electrostatic interactions (also called ionic bonding).

As noted above, prodrugs also fall within the scope of chemical entities, for example ester or amide derivatives of the compounds of Formula I. The term “prodrugs” includes any compounds that become compounds of Formula I when administered to a patient, e.g., upon metabolic processing of the prodrug. Examples of prodrugs include, but are not limited to, acetate, formate, and benzoate and like derivatives of functional groups (such as alcohol or amine groups) in the compounds of Formula I.

The term “solvate” refers to the compound formed by the interaction of a solvent, for example water or an alcohol, and a compound. Suitable solvates are pharmaceutically acceptable solvates, such as hydrates, including monohydrates and hemi-hydrates.

“Bond” refers to a covalent attachment between two atoms.

“Cyano” refers to the radical —CN.

“Cycloalkyl” refers to a saturated or unsaturated (although not aromatic) cyclic alkyl group. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. In certain embodiments, the cycloalkyl group can be C₃₋₁₀ cycloalkyl, such as, for example, C₃₋₆ cycloalkyl.

“Disease” refers to any disease, disorder, condition, symptom, or indication.

“Enzyme” refers to any naturally occurring or synthetic macromolecular substance composed wholly or largely of protein, that catalyzes, more or less specifically, one or more biochemical reactions. The substances upon which the enzyme acts are referred to “substrates,” for which the enzyme possesses a specific binding or “active site,” or “catalytic domain.” Enzymes can also act on macromolecular structures such as muscle fibers.

“Extended release” refers to dosage forms that provide for the delayed, slowed, over a period of time, continuous, discontinuous, or sustained release of the chemical entities of the present disclosure.

“Halogen” or “halo” refers to a fluoro, chloro, bromo, or iodo group.

“Heteroaryl” encompasses: 5- to 7-membered aromatic, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heterocycloalkyl aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Examples of heteroaryl groups include, but are not limited to, (as numbered from the linkage position assigned priority 1), 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzoimidazolinyl, indolinyl, pyridizinyl, triazolyl, quinolinyl, pyrazolyl, and 5,6,7,8-tetrahydroisoquinoline. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl as defined above. In certain embodiments, heteroaryl groups can be those derived from thiophene, pyrrole, benzothiophene, benzofuran, indole, pyridine, quinoline, imidazole, oxazole, pyrazine, benzothiazole, isoxazole, thiadiaxole, and thiazole.

“Heteroarylalkyl” or “heteroaralkyl” refers to an acyclic alkyl group in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl, and/or heteroarylalkynyl is used. In certain embodiments, the heteroarylalkyl group can be a 6 to 30 membered heteroarylalkyl, e.g., the alkanyl, alkenyl or alkynyl moiety of the heteroarylalkyl can be 1 to 10 membered and the heteroaryl moiety can be a 5 to 20-membered heteroaryl.

By “heterocycloalkyl” is meant a single aliphatic ring, usually with 3 to 7 ring atoms, containing at least 2 carbon atoms in addition to 1-3 heteroatoms independently selected from oxygen, sulfur, and nitrogen, as well as combinations comprising at least one of the foregoing heteroatoms. Suitable heterocycloalkyl groups include, for example (as numbered from the linkage position assigned priority 1), 2-pyrrolinyl, 2,4-imidazolidinyl, 2,3-pyrazolidinyl, 2-piperidyl, 3-piperidyl, 4-piperidyl, and 2,5-piperazinyl. Morpholinyl groups are also contemplated, including 2-morpholinyl and 3-morpholinyl (numbered wherein the oxygen is assigned priority 1). Substituted heterocycloalkyl also includes ring systems substituted with one or more oxo (=0) or oxide (—O⁻) substituents, such as piperidinyl N-oxide, morpholinyl-N-oxide, 1-oxo-1-thiomorpholinyl and 1,1-dioxo-1-thiomorpholinyl.

“Leaving group” refers to an atom or a group capable of being displaced by a nucleophile and includes halogen, such as chloro, bromo, fluoro, and iodo, alkoxycarbonyl (e.g., acetoxy), aryloxycarbonyl, mesyloxy, tosyloxy, trifluoromethanesulfonyloxy, aryloxy (e.g., 2,4-dinitrophenoxy), methoxy, N,O-dimethylhydroxylamino, and the like.

“Optional” or “optionally” means that the subsequently described event or circumstance may but need not occur, and that the description includes instances where the event or circumstance occurs and instances in which the event does not.

“Pharmaceutically acceptable” refers to approved or approvable by a regulatory agency of the Federal or a state government or listed in the U.S. Pharmacopeia or other generally recognized pharmacopeia for use in animals, and more particularly in humans.

“Pharmaceutically acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

“Pharmaceutically acceptable excipient, carrier or adjuvant” refers to an excipient, carrier or adjuvant that can be administered to a subject, together with at least one chemical entity of the present disclosure, and which does not destroy the pharmacological activity thereof and is nontoxic when administered in doses sufficient to deliver a therapeutic amount of the compound.

“Pharmaceutically acceptable vehicle” refers to a diluent, adjuvant, excipient or carrier with which at least one chemical entity of the present disclosure is administered.

“Prodrug” refers to a derivative of a therapeutically effective compound that requires a transformation within the body to produce the therapeutically effective compound. Prodrugs can be pharmacologically inactive until converted to the parent compound.

“Promoiety” refers to a form of protecting group that when used to mask a functional group within a drug molecule converts the drug into a prodrug. For example, the promoiety can be attached to the drug via bond(s) that are cleaved by enzymatic or non-enzymatic means in vivo.

“Protecting group” refers to a grouping of atoms that when attached to a reactive group in a molecule masks, reduces or prevents that reactivity. Examples of protecting groups can be found in Green et al., “Protective Groups in Organic Chemistry,” (Wiley, 2^{nd} ed. 1991) and Harrison et al., “Compendium of Synthetic Organic Methods,” Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”), and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Protein kinase,” “kinase,” and “human protein kinase” refer to any enzyme that phosphorylates one or more hydroxyl or phenolic groups in proteins, ATP being the phosphoryl-group donor.

“Stereoisomer” refers to an isomer that differs in the arrangement of the constituent atoms in space. Stereoisomers that are mirror images of each other and optically active are termed “enantiomers,” and stereoisomers that are not mirror images of one another are termed “diastereoisomers.”

“Subject” includes mammals, such as humans. The terms “human” and “subject” are used interchangeably herein.

“Substituted” refers to a group in which one or more hydrogen atoms are each independently replaced with the same or different substituent(s). Typical substituents include, but are not limited to, —X, —R³³, —O⁻, ═O, —OR³³, —SR³³, —S⁻, ═S, —NR³³R³⁴, ═NR³³, —CX₃, —CF₃, —CN, —OCN, —SCN, —NO, —NO₂, ═N₂, —N₃, —S(O)₂O⁻, —S(O)₂OH, —S(O)₂R³³, —OS(O₂)O⁻, —OS(O)₂R³³, —P(O)(O⁻)₂, —P(O)(OR³³)(O⁻), —OP(O)(OR³³)(OR³⁴), —C(O)R³³, —C(S)R³³, —C(O)OR³³, —C(O)NR³³R³⁴, —C(O)O⁻, —C(S)OR³³, —NR³⁵C(O)NR³³R³⁴, —NR³⁵C(S)NR³³R³⁴, —NR³⁵C(NR³³)NR³³R³⁴, —C(NR³³)NR³³R³⁴, —S(O)₂NR³³R³⁴, —NR³⁵S(O)₂R³³, —NR³⁵C(O)R³³, and —S(O)R³³ where each X is independently a halogen; each R³³ and R³⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, substituted heteroarylalkyl, —NR³⁵R³⁶, —C(O)R³⁵ or —S(O)₂R³⁵ or optionally R³³ and R³⁴ together with the atom to which R³³ and R³⁴ are attached form one or more cycloheteroalkyl, substituted cycloheteroalkyl, heteroaryl, or substituted heteroaryl rings; and R³⁵ and R³⁶ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl or substituted heteroarylalkyl, or optionally R³⁵ and R³⁶ together with the nitrogen atom to which R³⁵ and R³⁶ are attached form one or more cycloheteroalkyl, substituted cycloheteroalkyl, heteroaryl, or substituted heteroaryl rings. In certain embodiments, a tertiary amine or aromatic nitrogen may be substituted with one or more oxygen atoms to form the corresponding nitrogen oxide.

In certain embodiments, substituted aryl and substituted heteroaryl include one or more of the following substituent groups: F, Cl, Br, C₁₋₃ alkyl, substituted alkyl, C₁₋₃ alkoxy, —S(O)₂NR³³R³⁴, —NR³³R³⁴, —CF₃, —OCF₃, —CN, —NR³⁵S(O)₂R³³, —NR³⁵C(O)R³³, C₅₋₁₀ aryl, substituted C₅₋₁₀aryl, C₅₋₁₀ heteroaryl, substituted C₅₋₁₀ heteroaryl, C(O)OR³³, —NO₂, —C(O)R³³, —C(O)NR³³R³⁴, —OCHF₂, C₁₋₃ acyl, —SR³³, —S(O)₂OH, —S(O)₂R³³, —S(O)R³³, —C(S)R³³, —C(O)O⁻, —C(S)OR³³, —NR³⁵C(O)NR³³R³⁴, —NR³⁵C(S)NR³³R³⁴, and —C(NR³⁵)NR³³R³⁴, C₃₋₈ cycloalkyl, and substituted C₃₋₈ cycloalkyl, C₃₋₈ heterocycloalkyl, and substituted C₃₋₈ heterocycloalkyl, as defined herein.

In certain embodiments, substituted arylalkyl, and substituted heteroarylalkyl include one or more of the following substitute groups: F, Cl, Br, C₁₋₃ alkyl, C₁₋₃ alkoxy, —S(O)₂NR³³R³⁴, —NR³³R³⁴, —CF₃, —OCF₃, CN, —NR³⁵S(O)₂R³³, —NR³⁵C(O)R³³, C₅₋₁₀ aryl, substituted alkyl, substituted C₅₋₁₀ aryl, C₅₋₁₀ heteroaryl, substituted C₅₋₁₀ heteroaryl, —C(O)OR³³, —NO₂, —C(O)R³³, —C(O)NR³³R³⁴, —OCHF₂, C₁₋₃ acyl, —SR³³, —S(O)₂OH, —S(O)₂R³³, —S(O)R³³, —C(S)R³³, —C(O)O⁻, —C(S)OR³³, —NR³⁵C(O)NR³³R³⁴, —NR³⁵C(S)NR³³R³⁴, and —C(NR³⁵)NR³³R³⁴, C₃₋₈ cycloalkyl, and substituted C₃₋₈ cycloalkyl, as defined herein.

In certain embodiments, substituted alkyl includes one or more of the following substitute groups: C₁₋₃ alkoxy, —NR³³R³⁴, substituted C₅₋₁₀ heteroaryl, —SR³³, C₁₋₃ alkoxy, —S(O)₂ NR³³R³⁴, CN, F, Cl, —CF₃, —OCF₃, —NR³⁵S(O)₂R³³, —NR³⁵C(O)R³³, C₅₋₁₀ aryl, substituted C₅₋₁₀ aryl, C₅₋₁₀ heteroaryl, substituted C₅₋₁₀ heteroaryl, —C(O)OR³³, —NO₂, —C(O)R³³, —C(O)NR³³R³⁴, —OCHF₂, C₁₋₃ acyl, —S(O)₂OH, —S(O)₂R³³, —S(O)R³³, —C(S)R, —C(O)O⁻, —C(S)OR³³, —NR³⁵C(O)NR³³R³⁴, —NR³⁵C(S)NR³³R³⁴, and —C(NR³⁵)NR³³R³⁴, C₃₋₈ cycloalkyl, and substituted C₃₋₈ cycloalkyl, as defined herein.

In certain embodiments, substituted alkenyl includes one or more of the following substitute groups: C₁₋₈ alkyl, substituted C₁₋₈ alkyl, C₅₋₁₀ aryl, substituted C₅₋₁₀ aryl, C₅₋₁₀ heteroaryl, substituted C₅₋₁₀ heteroaryl, C₃₋₈ cycloalkyl, substituted C₃₋₈ cycloalkyl, cycloheteroalkylalkyl, and substituted cycloheteroalkylalkyl, as defined herein.

“Therapeutically effective amount” refers to the amount of a compound that, when administered to a subject for treating a disease, or at least one of the clinical symptoms of a disease or disorder, is sufficient to affect such treatment for the disease, disorder, or symptom and have a therapeutic effect. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation. The therapeutically effective amount reduces tumor size, activates complement, has apoptotic activity, or is capable of inducing cell death, and preferably death of benign or malignant tumor cells, in particular cancer cells. Efficacy can be measured in conventional ways, depending on the condition to be treated. For cancer therapy, efficacy can, for example, be measured by assessing the time for disease progression, survival, tumor size, or determining the response rates.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, and inhibit at least one physical parameter which may not be discernible to the subject. Further, “treating” or “treatment” refers to delaying the onset of the disease or disorder or at least symptoms thereof in a subject which may be exposed to or predisposed to a disease or disorder even though that subject does not yet experience or display symptoms of the disease or disorder.

In the specification and the appended claims, the singular forms “a,” “an,” and “the” include plural reference unless the context clearly dictates otherwise.

### 2-AMIDO-4-ISOXAZOLYL THIAZOLE COMPOUNDS

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

The compounds of Formula I can be named and numbered in the manner (e.g., using ChemDraw 8.0) described below. For example, compound 101:

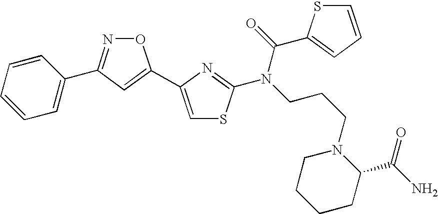

i.e., the compound according to Formula I where Q is thien-2-yl; A is 1,3-propylene, R¹ is 2-carbamoylpiperidin-1-yl, and R² is phenyl can be named (S)-1-(3-(N-(4-(3-phenylisoxazol-5-yl)thiazol-2-yl)thiophene-2-carboxamido)propyl)piperidine-2-carboxamide.

Provided is at least one chemical entity chosen from compounds of Formula I:

In certain embodiments, the compound of Formula I is chosen from
(S)-1-(3-(N-(4-(3-phenylisoxazol-5-yl)thiazol-2-yl)thiophene-2-carboxamido)propyl)piperidine-2-carboxamide;
(S)-1-(3-(N-(4-(3-phenylisoxazol-5-yl)thiazol-2-yl)thiophene-2-carboxamido)propyl)-N-hydroxypiperidine-2-carboxamide;
1-(3-(N-(4-(3-phenylisoxazol-5-yl)thiazol-2-yl)thiophene-2-carboxamido)propyl)piperidine-2-carboxamide;
(S)-1-(3-(N-(4-(3-phenylisoxazol-5-yl)thiazol-2-yl)thiophene-2-carboxamido)propyl)-N-(2-hydroxyethyl)piperidine-2-carboxamide;
(S)-1-(3-(N-(4-(3-phenylisoxazol-5-yl)thiazol-2-yl)thiophene-2-carboxamido)propyl)-N-methylpiperidine-2-carboxamide;
(S)-1-(3-(N-(4-(3-phenylisoxazol-5-yl)thiazol-2-yl)thiophene-2-carboxamido)propyl)pyrrolidine-2-carboxamide;
(S)-1-(3-(N-(4-(3-phenylisoxazol-5-yl)thiazol-2-yl)thiophene-2-carboxamido)propyl)-N,N-dimethylpiperidine-2-carboxamide;
1-(3-(N-(4-(3-phenylisoxazol-5-yl)thiazol-2-yl)thiophene-2-carboxamido)propyl)pyrrolidine-2-carboxamide;
(S)-1-(3-(N-(4-(3-phenylisoxazol-5-yl)thiazol-2-yl)thiophene-2-carboxamido)propyl)-N-methylpyrrolidine-2-carboxamide; and
(S)-17(3-(N-(4-(3-phenylisoxazol-5-yl)thiazol-2-yl)thiophene-2-carboxamido)propyl)-N-tert-butoxypiperidine-2-carboxamide.

Certain compounds of Formula I are potent inhibitors of AKT1 and also inhibit PIM1. In addition, the presence of the R³ group (i.e., a group of formula —C(O)NR⁴R⁵) increases the polarity of the compounds of Formula I. As such, the compounds of Formula I may exhibit improved physicochemical properties, e.g., solubility, may be easier to formulate than less polar compounds, and may exhibit improved pharmacokinetic properties when administered to patients, such as humans.

Certain 2-amido-thiazole compounds which are structurally related to the compounds of the present invention are described in US 2006/0052416 and demonstrated to have ATP-utilizing enzyme inhibitory activity, including AKT1 binding activity.

Protein kinases are among the largest and most functionally diverse gene families. Most of the over 500 human protein kinases belong to a single superfamily of enzymes in which the catalytic domains are related in sequence and structure. Most human protein kinases can further be grouped into seven major groups based on the deoxyribonucleic acid (DNA) sequence homologies identified as CAMK (calcium/calmodulin-dependent protein kinases), AGC (including PKA (protein kinase A), PKG (protein kinase G), PKC (protein kinase C) kinases), CK1 (casein kinases), CMGC (containing CDK (cyclin-dependent), MAPK (mitogen activated), GSK3 (glycogen synthase) and CLK (CDC2-like) kinases), STE (homologs of yeast Sterile 7, Sterile 11, and Sterile 20 kinases), TK (tyrosine kinases), and TKL (tyrosine-kinase like).

The AGC protein kinase family includes AKT1, AKT2, AKT3, AURORA-A, MSK1, MSK2, P70S6K, PAK1, PKA, and SGK1 protein kinases. The CMGC protein kinase family includes the CDK1, CDK2/cyclinA, CDK2/cyclinE, CDK5, DYRK2, GSK3-α, GSK3-β, P38-α, P38-β, P38-δ, and P38-γ, and MAPK1 protein kinases. The CAMK protein kinase family includes the DAPK1, MAPKAPK2, CHEK1, CHEK2, PRAK, and c-TAK1 protein kinases. The TK protein kinase family includes the ABL1, CSK, FLT3, FYN, HCK, INSR, KIT, LCK, PDGFR-α, LYNA, SYK, and SRC protein kinases. The STE protein kinase family includes PAK2 protein kinase.

Certain chemical entities of the present disclosure exhibited selectivity for one or more protein kinases, where selectivity is as defined herein. Certain chemical entities of the present disclosure exhibited selective activity for at least one of the following protein kinases: AKT1 and PIMN1 kinase. Certain chemical entities of the present disclosure exhibited selective activity for AKT1.

Chemical entities of the present disclosure can be prepared by methods well known in the art, including US 2006/0052416, and from readily available starting materials using the following general methods and procedures. It will be appreciated that where typical or preferred process conditions, such as, reaction temperatures, times, mole ratios of reactants, solvents, pressures, are given, other process conditions can also be used unless otherwise stated. Reaction conditions may vary with the reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, 3^{rd} Edition, John Wiley & Sons, (1999), and references cited therein.

Furthermore, chemical entities of the present disclosure can contain one or more chiral centers. Accordingly, if desired, such compounds can be prepared or isolated as pure stereoisomers, i.e., as individual enantiomers or diastereomers, or as stereoisomer-enriched mixtures. All such stereoisomers, and enriched mixtures thereof, are included within the scope of the present disclosure, unless otherwise indicated. Pure stereoisomers, and enriched mixtures thereof, can be prepared using, for example, optically active starting materials or stereoselective reagents well-known in the art. Alternatively, racemic mixtures of such compounds can be separated using, for example, chiral column chromatography, chiral resolving agents and the like.

General synthetic schemes and specific reaction protocols used to prepare chemical entities of the present disclosure are presented in the reaction schemes and Examples provided herein.

Chemical entities of the present disclosure can be prepared as shown in Scheme 1. Reaction of an α-haloketone 1 (e.g. X═Br or Cl) with an appropriately functionalized thiourea 2 can provide 2-amino-4-isoxazolylthiazoles 3. Acylation by conventional methods, preferably via the appropriate acid halide, can provide compounds of formula I. The required α-haloketones 1 can be prepared from the isoxazole acyl derivatives 4 by several methods. Where Y =Me, halogenation (e.g. bromination with Br₂) can provide compounds 1 directly. Carboxylic acids, where Y═OH, can be transformed into an acylating agent such as an acid chloride or a mixed acid anhydride, reacted with diazomethane to provide the diazoketone intermediate, which upon treatment with the appropriate mineral acid (e.g. HBr), can provide compounds 1 as well. Isoxazole acids or derivatives, including the methyl ketones, of various substitution patterns can be obtained commercially or are known in the art. For example, substituted and unsubstituted 4-phenylisoxazole-3-carboxylates can be prepared as given in U.S. Pat. No. 5,011,849; substituted and unsubstituted 3-phenylisoxazole-4-carboxylates can be prepared as given in U.S. Pat. No. 6,365,591 and references cited therein; substituted and unsubstituted 4-phenylisoxazole-5-carboxylates can be prepared as given in WO 97/27187; substituted and unsubstituted 3-phenylisoxazole-5-carboxylates can be prepared as given in U.S. Pat. No. 5,338,857 and Tet. Lett. (1983) 24:2193; substituted and unsubstituted 5-phenylisoxazole-3-carboxylates can be prepared as given in U.S. Pat. No. 3,752,819 and U.S. Pat. No. 6,884,821; and, substituted and unsubstituted 5-phenylisoxazole-4-carboxylates can be prepared as given in Tetrahedron (2002) 58:8581 and US 4243406. In addition, substituted and unsubstituted 1-(3-phenylisoxazol-5-yl)ethanones (4, Y═Me) can be prepared as given in EP 399645. Thioureas 2 can be prepared from the appropriate primary amine 6 via known procedures, such as reaction with thiophosgene followed by treatment of the resulting chloride with ammonia, reaction with FMOC-isothiocyanate followed by deprotection with piperidine, reaction with TMS-isocyanate followed by deprotection and thionation with Lawesson's reagent, or reaction with benzoyl isothiocyanate followed by acidic hydrolysis. Amines 6 can be prepared by alkylation of R¹H with the appropriate functionalized starting material 5, where X is a leaving group such as Cl, Br, I, or OMs, and P is an amine protecting group such as BOC, CBZ, or phthaloyl, followed by deprotection. Starting materials 5 are commercially available or may be prepared by those skilled in the art.

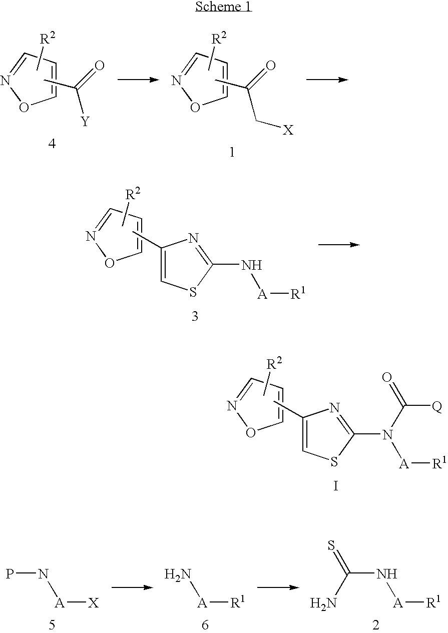

Compounds of the invention may also be prepared by procedures where the R¹ group is installed later in the synthetic sequence, as illustrated in Scheme 2. A functionalized amine such as 7 and 11 can be transformed into 2-amino-4-isoxazolylthiazoles 8 and 12, respectively, by procedures described above. Compound 9 (X=a leaving group such as Cl, Br, or OMs) can be prepared from alcohol 8 by sulfonylation or by sulfonylation followed by halogen displacement via methods known to one skilled in the art. Acylation to provide 10, followed by alkylation of the R¹H amine can give title compounds I. Conversion of 8 to 10 may also be accomplished by first acylation, followed by transformation of the OH functionality into a leaving group. Similarly, hydrolysis of 12 followed by acylation, or acylation followed by hydrolysis, can provide the aldehyde 13, which under reductive alkylation conditions, can give compounds of formula I.

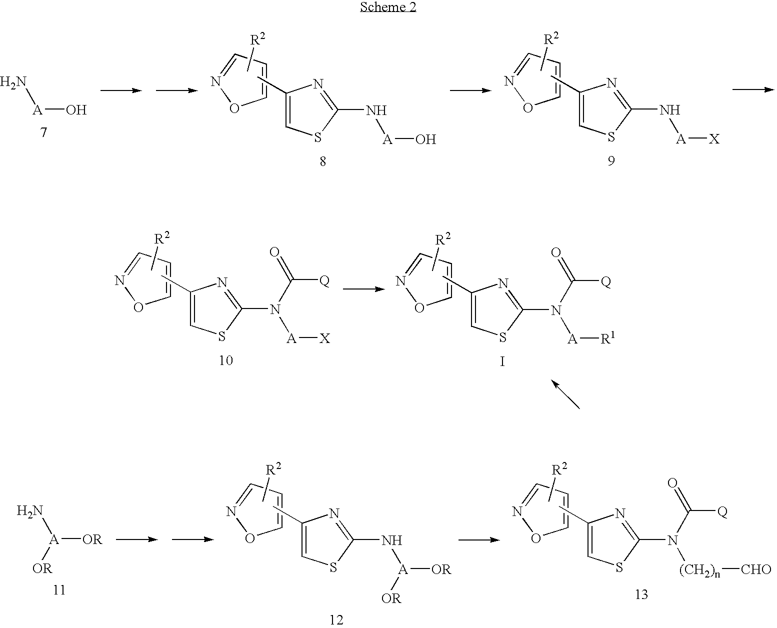

Alternatively, compounds of formula I may be prepared by routes where the isoxazole moiety is assembled later in the synthetic sequence as shown in Scheme 3. Synthesis of the starting aminothiazole ester 14 can arise from procedures described above from the appropriate starting materials. Reduction of the ester group to give the aldehyde 15 may occur directly by treatment with a reducing agent such as DIBAL at low temperatures (e.g. −78° C. to 0° C.), or by first complete reduction to the primary alcohol followed by oxidation. Reaction with hydroxylamnine can provide oxime 16, which may be treated with the appropriate alkyne and sodium hypochlorite, or via similar procedures referenced above, to yield the isoxazolylthiazole 17. Deprotection followed by acylation can then provide compounds of formula Ia where the isoxazolyl group is attached to the 4-position of thiazolyl at the 3-position of isoxazolyl, i.e. the carbon linked to nitrogen. Transformation of 15 into the acetylenic thiazole 18 can occur via methods known to one skilled in the art or described in Larock, R. C. Comprehensive Organic Transformations: A Guide to Functional Group Preparations, 2^{nd} ed.; Wiley & Sons: New York, (1999), pp 581-583, and references cited therein. Synthesis of the isoxazolylthiazole 19 can be achieved by reaction of the appropriate R² aldehyde with hydroxylamine to form an intermediate oxime, followed by reaction with 18 under cyclization conditions, such as with sodium hypochlorite, or via similar procedures referenced above. Deprotection followed by acylation can then provide compounds of formula Ib where the isoxazolyl group is attached to the 4-position of thiazolyl at the 2-position of isoxazolyl, i.e. the carbon linked to oxygen. Isomers Ia and Ib are compounds of the invention included in Formula I.

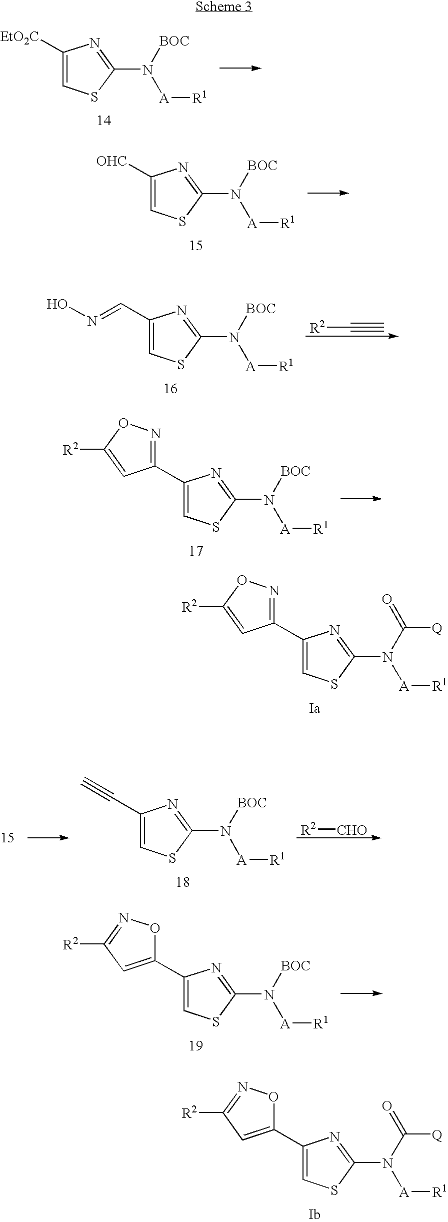

In accordance with certain embodiments, chemical entities of the present disclosure exhibit ATP-utilizing enzyme inhibitory activity. Thus, one important use of the chemical entities of the present disclosure includes the administration of at least one chemical entity of the present disclosure to a subject, such as a human. This administration serves to arrest, ameliorate, reduce the risk of acquiring, reduce the development of or at least one of the clinical symptoms of, or reduce the risk of developing or at least one of the clinical symptoms of diseases or conditions regulated by ATP-utilizing enzymes, such as, protein kinases.

For example, unregulated or inappropriately high protein kinase activity has been implicated in many diseases resulting from abnormal cellular function. Unregulated or inappropriately high protein kinase activity can arise either directly or indirectly, for example, by failure of the proper control mechanisms of a protein kinase, related, for example, to mutation, over-expression or inappropriate activation of the enzyme; or by over- or under-production of cytokines or growth factors also participating in the transduction of signal upstream or downstream of the protein kinase. In all of these instances, selective inhibition of the action of a protein kinase can be expected to have a beneficial effect.

According to certain embodiments, the present disclosure relates to methods of treating a disease regulated by at least one ATP-utilizing enzyme in a subject. ATP-utilizing enzyme regulated diseases include, for example, those where the ATP-utilizing enzyme participates in the signaling, mediation, modulation, control or otherwise involved in the biochemical processes affecting the manifestation of a disease. In certain embodiments, the methods are useful in treating diseases regulated by protein kinase enzymes. Protein kinase regulated diseases include, for example, the following general disease classes: cancer, autoimmunological, metabolic, inflammatory, infection, diseases of the central nervous system, degenerative neural disease, allergy/asthma, angiogenesis, neovascularization, vasculogenesis, cardiovascular, and the like. Without being limited by theory, specific examples of diseases that are known or believed to be regulated by protein kinase enzymes, include, transplant rejection, osteoarthritis, rheumatoid arthritis, multiple sclerosis, diabetes, diabetic retinopathy, asthma, inflammatory bowel disease such as Crohn's disease, and ulcerative colitis, renal disease cachexia, septic shock, lupus, diabetes mellitus, myasthenia gravis, psoriasis, dermatitis, eczema, seborrhea, Alzheimer's disease, Parkinson's disease, stem cell protection during chemotherapy, ex vivo selection or ex vivo purging for autologous or allogeneic bone marrow transplantation, leukemia including, but not limited to, acute myeloid leukemia, chronic myeloid leukemia, and acute lymphoblastic leukemia, cancer including but not limited to, breast cancer, lung cancer, colorectal cancer, ovarian cancer, prostate cancer, renal cancer, squamous cell cancer, glioblastoma, melanoma, pancreatic cancer, and Kaposi's sarcoma, ocular disease, comeal disease, glaucoma, bacterial infections, viral infections, fungal infections, heart disease, stroke, obesity, endometriosis, atherosclerosis, vein graft stenosis, peri-anastomatic prosthetic graft stenosis, prostate hyperplasia, chronic obstructive pulmonary disease, inhibition of neurological damage due to tissue repair, scar tissue formation, wound healing, pulmonary disease, neoplasm, macular degeneration.

Chemical entities of the present disclosure are particularly useful for the treatment of cancer including, but are not limited to, glioblastoma, ovarian cancer, breast cancer, endometrial carcinoma, hepatocellular carcinoma, melanoma, colorectal cancer, colon cancer, digestive tract, lung cancer, renal-cell carcinoma, thyroid, lymphoid, prostate cancer and pancreatic cancer, advanced tumors, hairy cell leukemia, melanoma, chronic myelogenous leukemia, advanced head and neck, squamous cell cancer, metastatic renal cell, non-Hodgkin's lymphoma, metastatic breast, breast adenocarcinoma, advanced melanoma, pancreatic, gastric, non-small cell lung, small cell lung, renal cell carcinoma, various solid tumors, multiple myeloma, metastatic prostate, malignant glioma, renal cancer, lymphoma, refractory metastatic disease, refractory multiple myeloma, cervical cancer, Kaposi's sarcoma, recurrent anaplastic glioma, and metastatic colon cancer.

More particularly, cancers that may be treated by chemical entities of the present disclosure, include, but are not limited to: Cardiac: sarcoma (angiosarcoma, fibrosarcoma, rhabdomyosarcoma, liposarcoma), myxoma, rhabdomyoma, fibroma, lipoma, teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous, cell carcinoma, adenocarcinoma, leiomyosarcoma, lymphoma) stomach (carcinoma, lymphoma, leiomyosarcoma), pancreas (ductal adenocarcinoma, insulinoma, glucagonoma, gastrinoma, carcinoid tumors, Karposi's sarcoma, leiomyoma, hemangioma, lipoma, neurofibroma, fibroma), large bowel (adenocarcinomas, tubular adenoma, villous adenoma, hamartoma, leiomyoma); Genitourinary tract: kidney (adenocarcinoma, Wilm's tumor[nephroblastoma], lymphoma, leukemia), bladder and uretha (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histocytoma, chondrosarcoma, Ewing's sarcoma, malignant lymphoma (reticulum cell sarcoma), multiple myeloma, malignant giant cell tumor chordoma, osteochronfroma(osteocartilaginous exostoses), benign chrodroma, chondroblastoma, chondromyxofibroma, osteoid osteoma and giant cell tumors; Nervous system: skull (osteoma, hemangioma, granuloma, xanthoma, osteitis deformans, meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenitial tumors), spinal cord, neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma], granulose-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma) vagina (clear cell carcinoma, squamous cell carcinoma, botryoid sarcoma (embryonal rhabdomyosarcoma), fallopian tubes carcinoma); Hematologic: blood (myeloid leukemia (acute and chronic], acute lymphoblastic leukemia, chronic lymphocytic leukemia, myeloproliferative diseases, multiple myeloma, myelodysplastic syndrome), Hodgkin's disease, non-Hodgkins's lymphoma [malignant lymphoma]; Skin: malignant melanoma, basel cell carcinoma, squamous cell carcinoma, Karposi's sarcoma, moles dysplastic nevi, lipoma, angioma, dermatofibroma, keloids, psoriasis; and Adrenal glands: neuroblastoma.

Chemical entities of the present disclosure may also be useful for the treatment of tuberous sclerosis complex.

Chemical entities of the present disclosure may also be useful for the treatment of other conditions (e.g., inflammatory disease), including, but are not limited to, rheumatoid arthritis, osteoarthritis, endometriosis, atherosclerosis, vein graft stenosis, peri-anastomatic prosthetic graft stenosis, prostate hyperplasia, chronic obstructive pulmonary disease, psoriasis, inhibition of neurological damage due to tissue repair, scar tissue formation, wound healing, multiple sclerosis, inflammatory bowel disease, infections, particularly bacterial, viral, retroviral or parasitic infections (by increasing apoptosis), pulmonary disease, neoplasm, Parkinson's disease, transplant rejection (as an immunosuppressant), macular degeneration and septic shock.

Chemical entities of the present disclosure may also be useful for the treatment of diseases mediated by, but not limited to, modulation or regulation of AKT protein kinases, tyrosine kinases, additional serine/threonine kinases, and/or dual specificity kinases.

In certain embodiments, a pharmaceutical composition can include at least one chemical entity of the present disclosure and at least one additional therapeutic agent appropriate for effecting combination therapy. Chemical entities of the present disclosure are also useful in combination with known therapeutic agents and anti-cancer agents. A person skilled in the art would be able to discern which combinations of agents would be useful based on the particular characteristics of the drugs and the cancer involved. Many chemotherapeutics are presently known in the art. Such anti-cancer agents include, but are not limited to, estrogen receptor modulators, cytostatic/cytotoxic agents, anti-proliferative agents, cell cycle checkpoint inhibitors, angiogenesis inhibitors, monoclonal antibody targeted therapeutic agents, tyrosine kinase inhibitors, serine-threonine kinase inhibitors, histone deacetylase inhibitors, heat shock protein inhibitors, and farnesyl transferase inhibitors. Chemical entities of the present disclosure are also useful in combination with radiation therapy.

Examples of cytostatic/cytotoxic agents, anti-proliferative agents and cell cycle checkpoint inhibitors include, but are not limited to, sertenef, cachectin, ifosfamide, tasonermin, lonidamine, carboplatin, altretarnine, prednimustine, dibro- modulcitol, ranimustine, fotemustine, nedaplatin, oxaliplatin, temozolomide, heptaplatin, estramustine, improsulfan tosilate, trofosfamide, nimustine, dibrospidium chloride, pumitepa, lobaplatin, satraplatin, profiromycin, cisplatin, irofulven, dexifosfamide, cis-aminedichloro(2-methylpyridine)platinum, benzylguanine, glufosfamide, GPXlOO, (trans, trans, trans)-bis-mu-(hexane-1,6-diamine)-mu [di-amine-platinum(II)]bis[diamine(chioro)platinum (II)]tetrachloride, diarizidinylspermine, arsenic trioxide,1-(11-dodecylamino-10-hydroxyundecyl)-3,7-dimethylxanthine, zocubicin, idarubicin, daunorubicin, bisantrene, mitoxantrone, pirarubicin, pinafide, valrubicin, amrubicin, antineoplaston, 3′-deamino-3′-morpholino-13-deoxo-10-hydroxy-carminomycin, annamycin, galarubicin, elioafide, MENI0755, and 4-demetboxy-3-deamino-3-aziridinyl-4-methylsulphonyl-daunorubicin.

An example of a hypoxia activatable compound is tirapazamine.

Examples of proteosome inhibitors include but are not limited to lactacystin and MLN-341 (Velcade).

Examples of microtubule inhibitors/microtubule-stabilizing agents include paclitaxel, vindesine sulfate, 3′,4′didehydro-4′-deoxy-8′norvincaleukoblastine, docetaxol, rhizoxin, dolastatin, mivobulin isethionate, auristatin, cemadotin, RPRI09881, BMS184476, vinflunine, and BMS188797.

Some examples of topoisomerase inhibitors are topotecan, bycaptamine, irinotecan, robitecan, 6-ethoxypropionyl-3′,4′O-exo-benzylidene-chartreusin.

“Inhibitors of kinases” involved in mitotic progression include, but are not limited to, inhibitors of aurora kinases, inhibitors of Polo-like kinases (PLK; in particular inhibitors of PLK-1), inhibitors of bub-1 and inhibitors of bub-Ri.

“Antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine. trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine.

Examples of monoclonal antibody targeted therapeutic agents include those therapeutic agents which have cytotoxic agents or radioisotopes attached to a cancer cell specific or target cell specific monoclonal antibody. Examples can be found in a number of references (Krause and Van Etten, 2005 New Eng. J. Med. 353,172184) and include, but are not limited to, Bexxar, trastuzumab (HERCEPTIN®), cetuximab (ERBITUX®), ABX-EGF, 2C4, bevacizumab (AVASTIN®), bortezomib (VELCADE®), rituximab (RITUXAN®).

Some specific examples of tyrosine inhibitors can be found in a number of references (Krause and Van Etten, 2005 New Eng. J. Med. 353,172184; Brown and Small 2004 Eur. J. Cancer 40,707-721; Fabian et al. 2005 Nat. Biotech. 23,329-336) and include imatinib (GLEEVEC®, ST1571), gefitnib (IRESSA®), BMS-354825, PKC412, PD 0173074, SU5402, MLN-518, CEP-701, SU5416, erlotinib (TARCEVA®), CI-1033, CT2923, sunitinib (SUTENT®, SU11248), GW-2016, EKB-569, ZD-6474, vatalanib (PTK-787), AMN107, ZD6474, CHIR-258, OSI-930, AZD0530, AEE788.

Some specific examples of serine/threonine kinase inhibitors can be found in a number of references (Jackman et al. 2004 Drug Disc Today: Ther. Strategies 1,445-454; Fabian et al. 2005 Nat. Biotech. 23,329-336; Pearson and Fabbro 2004, Expert Rev. Anticancer Ther. 4, 1113-1124) and include but are not limited to, LY-333531, sorafenib (BAY-43-9006), roscovitine (CYC202), CI-1040, ZM447439, CCI-779, RAD001, UNC01, VX680, AP23573.

Examples of heat shock protein inhibitors include, but are not limited to, 17-AAG and 17-DMAG.

Examples of histone deacetylase inhibitors include, but are not limited to, MS-275, AN-9, apicidin derivatives, Baceca, CBHA, CHAPs, chlamydocin, CS-00028, CS-055, EHT-0205, FK-228, FR-135313, G2M-777, HDAC-42, LBH-589, MGCD-0103, NSC-3852, PXD-101, pyroxamide, SAHA derivatives, suberanilohydroxamic acid, tacedinaline, VX-563, and zebularine.

Examples of farnesyl transferase inhibitors include, but are not limited to, lonafarnib.

Certain embodiments of the present disclosure are directed to methods of treating disease in a subject comprising the step of administering to a subject, in need of such treatment, a therapeutically effective amount of at least one chemical entity of the present disclosure. In some embodiments, a disease can be regulated by at least one ATP-utilizing enzyme such as a protein kinase. Certain diseases can be regulated by one or more ATP-utilizing enzymes. In such cases, treatment of the disease or disorder can include administering a therapeutically effective amount of at least one chemical entity of the present disclosure that inhibits the activity of one or more ATP-utilizing enzymes, or more than one compound of the present disclosure, wherein each compound inhibits at least one different ATP-utilizing enzyme.

Other embodiments of the present disclosure are related to methods of inhibiting at least one ATP-utilizing enzyme, including for example, a protein kinase. In certain embodiments, the ATP-utilizing enzyme can be inhibited by the method of administering to a subject, at least one chemical entity of the present disclosure, or a composition comprising at least one chemical entity of the present disclosure.

In certain embodiments, the present disclosure relates to methods of inhibiting ATP-utilizing enzyme activity by contacting at least one ATP-utilizing enzyme with at least one chemical entity of the present disclosure, such as the AKT-1 Kinase Assay (Example 6). ATP-utilizing enzymes include phosphotransferase enzymes that catalyze the phosphorylation of a biological molecule by transferring a phosphate group from an ATP substrate. ATP-utilizing enzymes include for example, synthetases, ligases, and kinases. Certain methods of the present disclosure are useful in inhibiting protein kinase enzymes, including, for example, the following protein kinase enzymes: AKT1 and PIM1 kinase. Certain methods of the present disclosure are useful in inhibiting AKT1.

Some methods of the present disclosure can be used to inhibit ATP-utilizing enzymes that are present in a living organism, such as a mammal; contained in a biological sample such as a cell, cell culture, or extract thereof, biopsied material obtained from a mammal or extracts thereof, and blood, saliva, feces, semen, tears or other body fluids or extracts thereof; contained within a reagent, or bound to a physical support. In certain embodiments, an ATP-utilizing enzyme can regulate a disease or disorder and in other embodiments, the ATP-utilizing enzyme may not regulate a disease or disorder.

According to the methods of the present disclosure, at least one ATP-utilizing enzyme can be inhibited by contact with at least one chemical entity of the present disclosure. In vivo ATP-utilizing enzymes can be inhibited by administration through routes and using compositions comprising at least one chemical entity of the present disclosure. For in vitro systems, contacting an ATP-utilizing enzyme with at least one chemical entity of the present disclosure can include, for example, combining liquid reagents or combining a reagent and an ATP-utilizing enzyme and/or compound of the present disclosure attached to a solid support. The ATP-utilizing enzyme and compound of the present disclosure can be contacted in any appropriate device such as an affinity chromatography column, a microarray, a microfluidic device, assay plate, or other appropriate chemical or biotechnology apparatus used to perform biochemical analysis, assay, screening, and the like, such as Example 6.

In certain embodiments, pharmaceutical compositions of the present disclosure may be administered orally, parenterally, by inhalation spray, topically, rectally, nasally, buccally, vaginally, via an implanted reservoir, or by any other appropriate route. Pharmaceutical compositions of the present disclosure can contain one or more pharmaceutically acceptable vehicles. In some embodiments, the pH of the formulation can be adjusted with pharmaceutically acceptable acids, bases or buffers to enhance the stability of the formulated compound or the delivery form. The term parenteral as used herein includes subcutaneous, intracutaneous, intravenous, intramuscular, intra-articular, intra-arterial, interasynovial, intrastemal, interathecal, intralesional, and intracranial injection or infusion techniques.

In certain embodiments, compounds disclosed herein can be delivered orally. Suitable dosage ranges for oral administration can depend on the potency of the compounds, but generally can range from 0.1 mg to 20 mg of a compound per kilogram of body weight. Appropriate dosages can be in the range of 25 to 500 mg/day and the dose of compounds administered can be adjusted to provide an equivalent molar quantity of compound in the plasma of a subject. Dosage ranges can be readily determined by methods known to those skilled in the art.

A dosage can be delivered in a composition by a single administration, by multiple applications, by sustained release or by controlled sustained release, or any other appropriate intervals and/or rates of release.

Chemical entities of the present disclosure can be assayed in vitro and in vivo, for the desired therapeutic or prophylactic activity prior to therapeutic use in mammals. For example, in vitro assays can be used to determine whether administration of a specific compound of the present disclosure or a combination of such compounds is effective for inhibiting the activity of certain ATP-utilizing enzymes or treating at least one disease. Chemical entities of the present disclosure can also be demonstrated to be effective and safe using animal model systems. A therapeutically effective dose of at least one chemical entity of the present disclosure can, in certain embodiments, provide therapeutic benefit without causing substantial toxicity. Toxicity of chemical entities of the present disclosure can be determined using standard pharmaceutical procedures and can be readily ascertained by the skilled artisan. The dose ratio between toxic and therapeutic effect is the therapeutic index. Chemical entities of the present disclosure can exhibit high therapeutic indices in treating diseases and disorders. The dosage of a compound of the present disclosure can be within a range of circulating concentrations that include an effective dose with little or no toxicity.

When employed as pharmaceuticals, chemical entities of the present disclosure can be administered in the form of pharmaceutical compositions. Such compositions can be prepared in a manner well known in the pharmaceutical art and can comprise at least one chemical entity of the present disclosure.

Pharmaceutical compositions of the present disclosure can comprise a therapeutically effective amount of at least one chemical entity of the present disclosure, and at least one pharmaceutically acceptable vehicle. Pharmaceutical compositions of the present disclosure can additionally comprise at least additional compound that enhances the therapeutic efficacy of one or more chemical entities of the present disclosure. For example, such compounds can enhance the therapeutic efficacy of chemical entities of the present disclosure by effectively increasing the plasma concentration of the compounds. Without being limited by theory, certain compound can decrease the degradation of the chemical entities of the present disclosure prior to administration or during transport to the plasma, or within the plasma. Certain compounds can increase the plasma concentration by increasing the absorption of compounds in the gastrointestinal tract. Pharmaceutical compositions of the present disclosure can also include additional therapeutic agents that are normally administered to treat a disease or disorder.

In certain embodiments, a pharmaceutical composition can include at least one chemical entity of the present disclosure and at least one additional therapeutic agent appropriate for effecting combination therapy.

In some embodiments, chemical entities and compositions of the present disclosure can be administered by oral routes. The compositions can be prepared in a manner well known in the pharmaceutical art and can comprise at least one chemical entity of the present disclosure. In some embodiments, compositions of the present disclosure contain a therapeutically effective amount of at least one chemical entity of the present disclosure, which can be in purified form, together with a therapeutically effective amount of at least one additional therapeutic agent, and a suitable amount of at least one pharmaceutically acceptable excipient, so as to provide the form for proper administration to a subject

Some embodiments of the present disclosure are directed to compositions that contain, as the active ingredient, of one or more chemical entities of the present disclosure associated with pharmaceutically acceptable excipients. In making certain compositions of the present disclosure, the active ingredient can be mixed with an excipient, diluted by an excipient, or enclosed within such a carrier that can be in the form of a capsule, sachet, paper or other container. When the excipient serves as a diluent, the excipient can be a solid, semi-solid, or liquid material, which acts as a vehicle, carrier or medium for the active ingredient. Thus, for example, the compositions can be in the form of tablets, pills, powders, lozenges, sachets, cachets, elixirs, suspensions, emulsions, solutions, and syrups containing, for example, from 1% to 90% by weight of at least one chemical entities of the present disclosure using, for example, soft and hard gelatin capsules.

In preparing a composition, it can be necessary to mill the active compound to provide the appropriate particle size prior to combining with other ingredients. If the active compound is insoluble, the active component ordinarily can be milled to a particle size of less than 200 mesh. If the active compound is water soluble, the particle size can be adjusted by milling to provide a uniform distribution in the formulation, e.g. 40 mesh.

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, modified cyclodextrins, cellulose, water, syrup, and methyl cellulose. Some compositions can additionally include, lubricating agents such as talc, magnesium stearate, and mineral oil, wetting agents, emulsifying and suspending agents, preserving agents such as methyl- and propylhydroxy-benzoates, sweetening agents, and flavoring agents. Compositions of the present disclosure can be formulated so as to provide quick, sustained or delayed release of the active ingredient after administration to the subject by employing procedures known in the art.

Some compositions of the present disclosure can be formulated in unit dosage form, each dosage containing, for example, 0.1 mg to 2 g of the active ingredient. As used herein, “unit dosage forms” refers to physically discrete units suitable as unitary dosages for human subjects and other mammals, each unit containing a predetermined quantity of active material calculated to produce the desired therapeutic effect, in association with a suitable pharmaceutical excipient, diluent, carrier and/or adjuvant. In certain embodiments, compositions of the present disclosure can be formulated in multiple dosage forms. The amount of the chemical entities of the present disclosure that can be combined with other materials and therapeutic agents to produce compositions of the present disclosure in a single dosage form will vary depending upon the subject and the particular mode of administration.

In the treatment of disease, chemical entities of the present disclosure can be administered in a therapeutically effective amount. It will be understood, however, that the amount of the compound administered will be determined by a physician, in the light of the relevant circumstances, including the condition to be treated, the chosen route of administration,. the actual compound administered, the age, weight, and response of the individual subject, the severity of the subject's symptoms, and the like.

For preparing solid compositions such as tablets, the principal active ingredient can be mixed with a pharmaceutical excipient to form a solid preformulation composition containing a homogeneous mixture of a compound of the present disclosure. When referring to these preformulation compositions as homogeneous, it is meant that the active ingredient is dispersed evenly throughout the composition so that the composition may be readily subdivided into equally effective unit dosage forms such as tablets, pills and capsules. The solid preformulation can then subdivided into unit dosage forms of the type described above containing from, for example, 0.1 mg to 2 g of the therapeutically effective compound of the present disclosure.

The tablets or pills comprising certain compositions of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials include a number of polymeric acids and mixtures of polymeric acids with such materials as shellac, cetyl alcohol, and cellulose acetate.

The liquid forms in which the compositions of the present disclosure may be incorporated for administration orally or by injection include aqueous solutions suitably flavored syrups, aqueous or oil suspensions, and flavored emulsions with edible oils such as cottonseed oil, sesame oil, coconut oil, or peanut oil, as well as elixirs and similar pharmaceutical vehicles.

As used herein, a “pharmaceutically acceptable derivative or prodrug” refers to any pharmaceutically acceptable salt, ester, salt of an ester or other derivative of a compound of the present disclosure that, upon administration to a recipient, is capable of providing, either directly or indirectly, a compound of the present disclosure or an inhibitory active metabolite or residue thereof. Examples of such derivates or prodrugs include those that increase the bioavailability of the chemical entities of the present disclosure when such compounds are administered to a mammal, e.g., by allowing an orally administered compound to be more readily absorbed into the blood, or which enhance delivery of the parent compound to a biological compartment, e.g., the brain or lymphatic system, relative to the parent species.

In certain embodiments, acceptable formulation materials can be nontoxic to recipients at the dosages and concentrations employed.

In certain embodiments, a pharmaceutical composition of the present disclosure can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids such as glycine, glutamine, asparagine, arginine or lysine; antimicrobials; antioxidants such as ascorbic acid, sodium sulfite, or sodium hydrogen-sulfite; buffers such as borate, bicarbonate, Tris-HCl, citrates, phosphates or other organic acids; bulking agents such as mannitol or glycine; chelating agents such as ethylenediamine tetraacetic acid (EDTA); complexing agents such as caffeine, polyvinylpyrrolidone, beta-cyclodextrin, hydroxypropyl-beta-cyclodextrin, or sulfobutyl ether ′cyclodextrin; fillers; monosaccharides; disaccharides; and other carbohydrates such as glucose, mannose, or dextrins; proteins such as serum albumin, gelatin or immunoglobulins; coloring, flavoring and diluting agents; emulsifying agents; hydrophilic polymers such as polyvinylpyrrolidone; low molecular weight polypeptides; salt-forming counter ions such as sodium; preservatives such as benzalkonium chloride, benzoic acid, salicylic acid, thimerosal, phenethyl alcohol, methylparaben, propylparaben, chlorhexidine, sorbic acid or hydrogen peroxide; solvents such as glycerin, propylene glycol or polyethylene glycol; sugar alcohols such as mannitol or sorbitol; suspending agents; surfactants or wetting agents such as pluronics, PEG, sorbitan esters, polysorbates such as polysorbate 20, polysorbate 80, triton, tromethamine, lecithin, cholesterol, tyloxapal; stability enhancing agents such as sucrose or sorbitol; tonicity enhancing agents such as alkali metal halides, such as sodium or potassium chloride, mannitol, sorbitol; delivery vehicles; diluents; excipients and/or pharmaceutical adjuvants (Remington's Pharmaceutical Sciences, 18^{th} Edition, A. R. Gennaro, ed., Mack Publishing Company (1990)).

In certain embodiments, the optimal pharmaceutical composition can be determined by one skilled in the art depending upon, for example the intended route of administration, delivery format, and desired dosage. See, for example, Remington's Pharmaceutical Sciences, supra. In certain embodiments, such compositions may influence the physical state, stability, rate of in vivo release, and rate of in vivo clearance of the antibodies of the present disclosure.

In certain embodiments, the primary vehicle or carrier in a pharmaceutical composition can be either aqueous or non-aqueous in nature. For example, in certain embodiments, a suitable vehicle or carrier can be water for injection, physiological saline solution or artificial cerebrospinal fluid, possibly supplemented with other materials common in compositions for parenteral administration. In certain embodiments, neutral buffered saline or saline mixed with serum albumin are further exemplary vehicles. In certain embodiments, pharmaceutical compositions comprise Tris buffer of pH 7 to 8.5, or acetate buffer of pH 4 to 5.5, which can further comprise sorbitol or a suitable substitute thereof. In certain embodiments, buffers are used to maintain the composition at physiological pH or at a slightly lower pH, typically within a pH range of from 5 to 8.

In certain embodiments, pharmaceutical compositions of the present disclosure can be selected for parenteral delivery. In other embodiments, compositions can be selected for inhalation or for delivery through the digestive tract, such as orally. The preparation of such pharmaceutically acceptable compositions is within the skill of the art.

In certain embodiments, composition components can be present in concentrations that are acceptable to the site of administration. In certain embodiments, when parenteral administration is contemplated, a therapeutic composition can be in the form of a pyrogen-free, parenterally acceptable aqueous solution comprising at least one chemical entity of the present disclosure, with or without additional therapeutic agents, in a pharmaceutically acceptable vehicle. In other embodiments, a vehicle for parenteral injection can be sterile distilled water in which at least one chemical entity of the present disclosure, with or without at least one additional therapeutic agent, is formulated as a sterile, isotonic solution, properly preserved. In still other embodiments, the pharmaceutical composition can include encapsulation of at least one chemical entity of the present disclosure with an agent, such as injectable microspheres, bio-erodible particles, polymeric compounds such as polyacetic acid or polyglycolic acid, beads or liposomes, that can provide the controlled or sustained release of the compound of the present disclosure which can then be delivered via a depot injection. In certain embodiments, implantable drug delivery devices can be used to introduce a compound of the present disclosure to the plasma of a subject, within a target organ, or to a specific site within the subject's body.

In certain embodiments, a pharmaceutical composition can be formulated for inhalation. In certain embodiments, a compound of the present disclosure, with or without at least one additional therapeutic agent, can be formulated as a dry powder for inhalation. In certain embodiments, an inhalation solution comprising a compound of the present disclosure with or without at least one additional therapeutic agent can be formulated with a propellant for aerosol delivery. In other embodiments, solutions can be nebulized. In still other embodiments, solutions, powders or dry films of chemical entities of the present disclosure can be aerosolized or vaporized for pulmonary delivery.

In certain embodiments, it is contemplated that formulations can be administered orally. In certain embodiments, a compound of the present disclosure, with or without at least one additional therapeutic agent that can be administered orally, can be formulated with or without carriers customarily used in the compounding of solid dosage forms such as tablets and capsules. In other embodiments, a capsule may be designed to release the active portion of the formulation in the region of the gastrointestinal tract where bioavailability can be maximized and pre-systemic degradation minimized. In still other embodiments, at least one additional agent can be included in the formulation to facilitate absorption of the compound of the present disclosure and/or any additional therapeutic agents into the systemic circulation. In certain embodiments, diluents, flavorings, low melting point waxes, vegetable oils, lubricants, suspending agents, tablet disintegrating agents, and binders can be employed.

In certain embodiments, a pharmaceutical composition of the present disclosure can include an effective quantity of chemical entities of the present disclosure, with or without at least one additional therapeutic agent, in a mixture with at least one pharmaceutically acceptable vehicle suitable for the manufacture of tablets. In certain embodiments, by dissolving the tablets in sterile water, or other appropriate vehicle, solutions can be prepared in unit-dose form. In certain embodiments, suitable excipients include inert diluents, such as calcium carbonate, sodium carbonate or bicarbonate, lactose, or calcium phosphate; or binding agents, such as starch, gelatin, or acacia; and lubricating agents such as magnesium stearate, stearic acid or talc.

In certain embodiments, the frequency of dosing will take into account the pharmacokinetic parameters of the chemical entities of the present disclosure and/or any additional therapeutic agents in the pharmaceutical composition used. In certain embodiments, a clinician can administer the composition until a dosage is reached that achieves the desired effect. The composition can be administered as a single dose, or as two or more doses, which may or may not contain the same amount of the therapeutically active compound time, or as a continuous infusion via an implantation device or catheter. Further refinement of an appropriate dosage can be routinely made by those of ordinary skill in the art. For example, therapeutically effective amounts and regimens can be determined through use of appropriate dose-response data.

In certain embodiments, the route of administration of the pharmaceutical composition can be in accord with known methods, e.g. orally, through injection by intravenous, intraperitoneal, intracerebral (intra-parenchymal), intracerebroventricular, intramuscular, intra-ocular, intraarterial, intraportal, or intralesional routes; by sustained release systems or by implantation devices. In certain embodiments, the compositions can be administered by bolus injection or continuously by infusion, or by an implantation device.

In certain embodiments, the composition can be administered locally via implantation of a membrane, sponge or another appropriate material onto which the desired compound of the present disclosure has been absorbed or encapsulated. In certain embodiments, where an implantation device is used, the device can be implanted into any suitable tissue or organ, and delivery of the desired molecule via diffusion, timed-release bolus, or continuous administration.

In certain embodiments, it can be desirable to use a pharmaceutical composition comprising a compound of the present disclosure, with or without at least one additional therapeutic agent, in an ex vivo manner. For example, cells, tissues and/or organs that have been removed from a subject are exposed to a pharmaceutical composition comprising a compound of the present disclosure, with or without at least one additional therapeutic agent, after which the cells, tissues and/or organs are subsequently implanted back into the subject.

Pharmaceutical compositions according to the present disclosure can take a form suitable for oral, buccal, parenteral, nasal, topical or rectal administration, or a form suitable for administration by inhalation or insufflation.

The compositions of the present disclosure can, if desired, be presented in a pack or dispenser device that can contain one or more unit dosage forms containing the active ingredient. The pack or dispensing device can be accompanied by instructions for administration.

The quantity of a compound of the present disclosure required for the treatment of a particular condition can vary depending on the compound, and the condition of the subject to be treated. In general, daily dosages can range from 100 ng/kg to 100 mg/kg, e.g., 0.01 mg/kg to 40 mg/kg body weight, for oral or buccal administration; from 10 ng/kg to 50 mg/kg body weight, e.g., 0.001 mg/kg to 20 mg/kg body weight, for parenteral administration; and from 0.05 mg to 1,000 mg for nasal administration or administration by inhalation or insufflation.

Certain chemical entities of the present disclosure and/or compositions of the present disclosure can be administered as sustained release systems. In certain embodiments, the chemical entities of the present disclosure can be delivered by oral sustained release administration. In this embodiment, the chemical entities of the present disclosure can be administered, for example, twice per day and, once per day.

The chemical entities of the present disclosure can be practiced with a number of different dosage forms, which can be adapted to provide sustained and/or extended release of a compound upon oral administration. Examples of sustained and/or extended release dosage forms include, but are not limited to, beads comprising a dissolution or diffusion release composition and/or structure, an oral sustained release pump, enteric-coated preparations, compound-releasing lipid matrices, compound releasing waxes, osmotic delivery systems, biodegradable polymer matrices, diffusible polymer matrices, a plurality of time-release pellets, and osmotic dosage forms.

Regardless of the specific form of sustained release oral dosage form used, the compounds and composition of the present disclosure can be released from the dosage form over an extended period of time. In certain embodiments, sustained release oral dosage forms can provide a therapeutically effective amount of a compound of the present disclosure over a period of at least several hours. In certain embodiments the extended release dosage form can provide a constant therapeutically effective concentration of a compound of the present disclosure in the plasma of a subject for a prolonged period of time, such as at least several hours. In other embodiments, the sustained release oral dosage form can provide a controlled and constant concentration of a therapeutically effective amount of a compound of the present disclosure in the plasma of a subject.

Dosage forms comprising compositions and chemical entities of the present disclosure can be administered at certain intervals such as, for example, twice per day or once per day.

Exemplary dosage ranges for oral administration are dependent on the potency of the compound of the present disclosure, but can range from 0.1 mg to 20 mg of the compound per kilogram of body weight. Dosage ranges may be readily determined by methods known to those skilled in the art.

Also provided are packaged pharmaceutical formulations. Such packaged formulations include a pharmaceutical composition comprising at least one chemical entity of the present disclosure, and instructions for using the composition to treat a mammal (typically a human patient). In some embodiments, the instructions are for using the pharmaceutical composition to treat a patient suffering from a disease responsive to inhibition at least one ATP-utilizing enzyme, such as a human protein kinase, for example AKT1 and PIM1 kinase. Also provided is prescribing information; for example, to a patient or health care provider, or as a label in a packaged pharmaceutical formulation. Prescribing information may include for example efficacy, dosage and administration, contraindication and adverse reaction information pertaining to the pharmaceutical formulation.

Chemical entities of the present disclosure can be assayed in vitro and in vivo, to determine and optimize therapeutic or prophylactic activity prior to use in subjects. For example, in vitro assays can be used to determine whether administration of a specific compound of the present disclosure or a combination of such compounds exhibits therapeutic efficacy. Chemical entities of the present disclosure can also be demonstrated to be effective and safe using animal model systems.

It is desirable that a therapeutically effective dose of a compound of the present disclosure provide therapeutic benefit without causing substantial toxicity. Toxicity of chemical entities of the present disclosure can be determined using standard pharmaceutical procedures and can be readily ascertained by the skilled artisan. The dose ratio between toxic and therapeutic effect is the therapeutic index. In certain embodiments, chemical entities of the present disclosure can exhibit particularly high therapeutic indices in treating diseases and disorders. In certain embodiments, the dosage of a compound of the present disclosure can be within a range of circulating concentration that exhibits therapeutic efficacy with limited or no toxicity.

### EXAMPLES

Embodiments of the present disclosure can be further defined by reference to the following examples, which describe in detail preparation of chemical entities of the present disclosure and assays for using chemical entities of the present disclosure. It will be apparent to those skilled in the art that many modifications, both to materials and methods, may be practiced without departing from the scope of the present disclosure. If an abbreviation is not defined, it has its generally accepted meaning.

### Example 1

### (S)—N,N-dimethylpiperidine-2-carboxamide hydrochloride

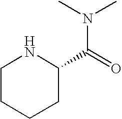

To a stirred suspension of (S)-(−)-1-Boc-2-piperidinecarboxylic acid (750 mg, 3.2 mmol), BOP reagent (benzotriazole-1-yl-oxy-tris-(dimethylamino)-phosphonium hexafluorophosphate) (2.16 g, 4.9 mmol), and dimethylamine hydrochloride (399 mg, 4.9 mmol) in acetonitrile (50 mL) was added triethylamine (0.9 mL, 6.5 mmol) The reaction mixture was stirred at room temperature overnight then concentrated in vacuo. The resulting residue was taken up into ethyl acetate (150 mL) and washed with saturated potassium hydrogen sulfate (150 mL), saturated sodium bicarbonate (150 mL) and brine (150 mL). The organics were dried with sodium sulfate, filtered and concentrated in vacuo. The resulting residue was purified by flash chromatography on silica gel (10 g) eluting with ethyl acetate/hexanes (2:1) to give (S)-tert-butyl-2-(dimethoxycarbamoyl)piperidine-1-carboxylate (700 mg) as a clear viscous oil.

To a solution of (S)-tert-butyl-2-(dimethoxycarbamoyl)piperidine-1-carboxylate (700 mg, 2.73 mmol) in ethyl acetate (10 mL) was added 2N HCl in diethyl ether (10 mL). The reaction mixture was allowed to stir at room temperature for 12 h, after which time the title compound had precipitated from the reaction mixture as a gum. The supernatant was decanted and the residues were triturated with diethyl ether (2×50 mL) to give the title compound as a white solid. This and other resulting amine hydrochlorides prepared similarly were used directly in subsequent reactions.

### Example 2

### (S)-1-(3-(N-(4-(3-phenylisoxazol-5-yl)thiazol-2-yl)thiophene-2-carboxamido)propyl)piperidine-2-carboxamide hydrochloride 101

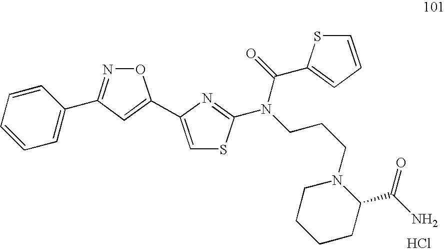

A mixture of 1-amino-3,3-diethoxypropane (13.1 g, 88.8 mmol) and 9-fluorenylmethoxycarbonyl isothiocyanate (25.0 g, 88.8 mmol) in dry chloroform (200 mL) was stirred at room temperature for 2 h, after which time TLC analysis (4:1 hexanes/ethyl acetate) indicated the reaction was complete. The reaction was concentrated in vacuo and the resulting residue was suspended in ethyl acetate (400 mL) followed by the addition of piperidine (13.2 mL, 133.0 mmol). After stirring at room temperature for 15 h, the reaction was concentrated in vacuo. The resulting residue was chromatographed on silica gel (800 mL) eluting with a gradient mobile phase of hexanes/ethyl acetate (3:1 to 0:100). All fractions containing the desired product were combined and concentrated in vacuo to give 1-(3,3-diethoxypropyl)thiourea (17.6 g) as a viscous orange oil that slowly crystallized upon standing.

¹H NMR (300 MHz, CDCl₃) δ 6.71 (br s, 1H); 6.08 (br s, 1H); 5.55 (br s, 1H); 4.61 (t, 1H); 3.68 (m, 2H); 3.52 (m, 2H), 3.28 (br s, 2H); 1.92 (m, 2H); 1.24 (t, 6H).

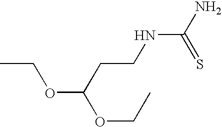

A mixture of 1-(3,3-diethoxypropyl)thiourea (15.5 g, 75.1 mmol) and diisopropylethylamine (26.2 mL, 150.5 mmol) was prepared in dioxane (500 mL) to which was added 2-bromo-1-(3-phenylisoxazol-5-yl)ethanone (20.0 g, 75.1 mmol). The reaction mixture was stirred 80° C. for 1 h, after which time TLC analysis (1:1 hexanes/ethyl acetate) indicated the reaction was complete. The solvents were removed in vacuo, and the residue was taken up in ethyl acetate (200 mL) and washed with saturated aqueous sodium bicarbonate (100 mL) and saturated aqueous sodium chloride (100 mL). The organics were dried over sodium sulfate, filtered, and concentrated in vacuo to give a brown-tan solid. The residue was taken up in hot acetonitrile (150 mL) and the desired product crystallized as a light tan solid. The crystals were washed with cold acetonitrile to afford N-(3,3-diethoxypropyl)-4-(3-phenylisoxazol-5-yl)thiazol-2-amine (14.1 g) as a light tan crystalline solid (mp 92-3° C.). ¹H NMR (300 MHz, CDCl₃) δ 7.86 (m, 2H); 7.46 (m, 3H); 7.09 (s, 1H); 6.87 (s, 1H); 5.80 (m, 1H); 4.66 (t, 1H); 3.71 (pent, 2H); 3.52 (pent, 2H); 3.45 (m, 2H); 2.01 (m, 2H); 1.26 (t, 6H). TLC analysis (2:1 hexanes/ethyl acetate) R_{f}=0.44.

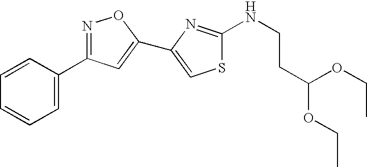

A 20 mL glass microwave reaction tube was charged with N-(3,3-diethoxypropyl)-4-(3-phenylisoxazol-5-yl)thiazol-2-amine (2.0 g, 5.3 mmol), chloroform (7 mL), diisopropylethylamine (5.0 mL) and thiophene-2-carbonyl chloride (2.0 mL, 18.7 mmol). The reaction was heated for 1600 s at 140° C. A total of seven identical reactions were conducted, processing a total of 14 g of the starting amine. The combined crude reaction mixtures were washed with saturated aqueous potassium hydrogen sulfate (200 mL), saturated aqueous sodium bicarbonate (200 mL), and saturated aqueous sodium chloride (200 mL). The organics were dried over sodium sulfate, filtered, and concentrated in vacuo to give the crude product as a brown oil. The residue was chromatographed on silica gel (600 mL) eluting with a gradient mobile phase of hexanes/ethyl acetate (5:95 to 20:80) to give a yellow tacky semi-solid. The resulting material was triturated with diethyl ether (200 mL) to give N-(3,3-diethoxypropyl)-N-(4-(3-phenylisoxazol-5-yl)thiazol-2-yl)thiophene-2-carboxamide (15 g) as a cream colored solid (mp 100-1° C). ¹H NMR (300 MHz, CDCl₃) δ 7.88 (m, 3H); 7.67 (m, 1H); 7.62 (s, 1H); 7.49 (m, 3H); 7.17 (m, 1H); 6.90 (s, 1H); 4.67 (m, 3H); 3.72 (pent, 2H); 3.55 (pent, 2H); 2.35 (m, 2H); 1.25 (t, 6H). TLC analysis (2:1 hexanes/ethyl acetate) R_{f}=0.62.

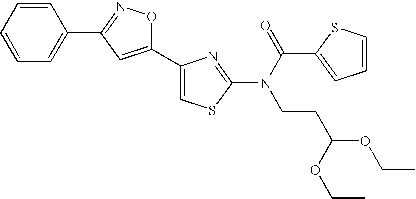

A mixture of N-(3,3-diethoxypropyl)-N-(4-(3-phenylisoxazol-5-yl)thiazol-2-yl)thiophene-2-carboxamide (8.0 g) in dioxane (70 mL) at 0° C. was treated with HCl in diethyl ether (2M, 70 mL). After stirring for 1.5 h at room temperature, the resulting suspension was carefully quenched with saturated aqueous sodium bicarbonate (300 mL), and the product was extracted with diethyl ether (2×150 mL). The combined organics were dried over sodium sulfate, filtered, and concentrated in vacuo. The resulting solids were triturated with diethyl ether (100 mL) to give N-(3-oxopropyl)-N-(4-(3-phenylisoxazol-5-yl)thiazol-2-yl)thiophene-2-carboxamide (12.6 g) as a cream colored solid. ¹H NMR (300 MHz, CDCl₃) δ 9.95 (s, 1H); 7.91 (m, 2H); 7.68 (m, 1H); 7.62 (s, 1H); 7.58 (m, 1H); 7.78 (m, 2H); 7.19 (m, 2H); 6.92 (s, 1H); 4.85 (t, 2H); 3.22 (t, 2H). TLC analysis (2:1 hexanes/ethyl acetate) R_{f}=0.36. LC/MS (APCI) m/z 410.2 [M+H].

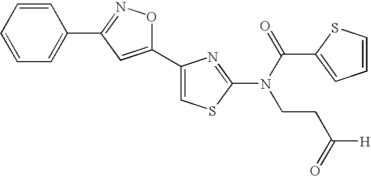

A mixture of N-(3-oxopropyl)-N-(4-(3-phenylisoxazol-5-yl)thiazol-2-yl)thiophene-2-carboxamide (6.0 g, 14.6 mmol) and glacial acetic acid (30 mL) in 1,2-dichloroethane (90 mL) was stirred at room temperature. In a second reaction vessel was charged (S)-piperidine-2-carboxamide (1.8 g, 15.8 mmol) and glacial acetic acid (30 mL) in 1,2-dichloroethane (90 mL). After 10 min of stirring, sodium triacetoxyborohydride (4.6 g, 22.0 mmol) was then added to the amine solution. The amine solution was then added directly to the flask containing the aldehyde substrate and stirred at room temperature for 10 min. The reaction mixture was quenched with water (100 mL) and washed with saturated aqueous potassium hydrogen sulfate (150 mL), saturated aqueous sodium bicarbonate (150 mL), dried over sodium sulfate, filtered, and concentrated in vacuo to give a pale yellow residue. The crude material was purified by chromatography on silica gel (600 mL) eluting with a gradient mobile phase of dichloromethane (100%) to ethyl acetate (100%) and finally methanol/ethyl acetate (5:95). All fractions containing the desired product were combined and concentrated in vacuo to give a white solid. The free amine was dissolved in dichloromethane (30 mL) to which was added HCl in diethyl ether (2M, 40 mL), which resulted in an immediate precipitate formation. After 30 min the supernatant was decanted from the resulting solids and the residue was triturated with diethyl ether (200 mL). The solids were collected and dried overnight in vacuo. The resulting solids were dissolved in a mixture of acetonitrile/water (50 mL, 1:1 v/v) and lyophilized resulting in (S)-1-(3-(N-(4-(3-phenylisoxazol-5-yl)thiazol-2-yl)thiophene-2-carboxamido)propyl)piperidine-2-carboxamide hydrochloride 101 (5.8 g) as a lyophilate. ¹H NMR (300 MHz, CD₃OD) δ 7.94 (m, 2H); 7.86 (m, 1H), 7.83 (s, 1H); 7.71 (m, 1H); 7.49 (m, 3H); 7.29 (s, 1H); 7.20 (m, 1H); 4.52 (m, 2H); 3.91 (br d, 1H); 3.79 (br d, 1H); 3.35 (m, 1H); 3.17 (br t, 1H); 2.51 (m, 2H); 2.22 (br d, 1H); 2.02-1.75 (m, 3H); 1.64 (m, 1H). TLC analsis (93:6:1 chloroform/methanol/concentrated ammonium hydroxide) R_{f}=0.46. LC/MS (APCI) m/z 522.4 [M+H]. HPLC retention time (Method A)=5.92 min. [α]_{D}²⁵−15° (c 1.0, MeOH).

### Example 3

### 1-(3-(N-(4-(3-phenylisoxazol-5-yl)thiazol-2-yl)thiophene-2-carboxamido)propyl)pyrrolidine-2-carboxamide 102

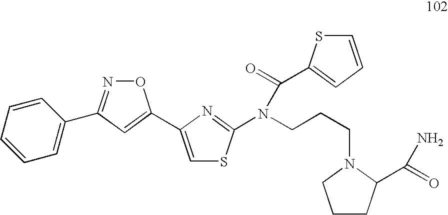

A solution of 3-amino-1-propanol (1.67 mL, 22 mmol) in chloroform (100 mL) was treated with 4 Å molecular sieves (3 g) for 72 h, filtered, and FMOC-isothiocyanate (6.18 g, 22 mmol) was added to the filtrate. The reaction mixture was stirred at room temperature for 1 h, then concentrated in vacuo. The resulting residue was dissolved in EtOAc (100 mL) and piperidine (3.27 mL, 33 mmol) was added. The reaction mixture was maintained at ambient temperature for 30 min, then the formed precipitate was filtered, washed with EtOAc and dried in vacuo to provide 1-(3-hydroxypropyl)thiourea (2.42 g) as a white solid.

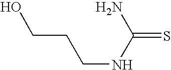

A mixture of 2-bromo-1-(3-phenylisoxazol-5-yl)ethan-1-one (795 mg, 2.99 mmol) and the thiourea prepared above (400 mg, 2.99 mmol) was dissolved in dry dioxane (8 mL). The reaction mixture was heated at 80° C. for 2 h, cooled to room temperature, and then concentrated in vacuo. The formed precipitate was filtered, washed with dioxane and dissolved in chloroform (50 mL). The mixture was washed with 5% aqueous Na₂CO₃ and brine, dried over MgSO₄ and evaporated in vacuo. The resulting residue was crystallized from the mixture of ether/hexane (1:5) to provide 3-(4-(3-phenylisoxazol-5-yl)thiazol-2-ylamino)propan-1-ol (681 mg) as an off-white solid.

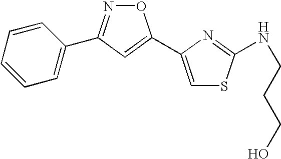

A mixture of the aminothiazole prepared above (671 mg, 2.23 mmol) and N,O-bis(trimethylsilyl)acetamide (1.1 mL, 4.46 mmol) was dissolved in dry chloroform (30 mL). The mixture was heated in a pressure vessel at 80° C. for 30 min. and cooled to the room temperature. N,N-Diisopropylethylamine (1.55 mL, 8.92 mmol) was added, followed by the addition of 2-thiophenecarbonyl chloride (572 μL, 5.36 mmol). The reaction mixture was irradiated in a microwave oven (max. power 250W, 120° C.) for 30 min and cooled to ambient temperature. The resulting solution was washed with water (30 mL×2) and concentrated in vacuo. The resulting residue was dissolved in DMSO (2 mL) and subjected to HPLC purification (YMC-Pack ODS-A C-18 column (30 mm×100 mm); flow rate=45 mL/min; injection volume=2 mL; mobile phase A: 100% water, 0.1% trifluoroacetic acid (TFA); mobile phase B: 100% acetonitrile, 0.1% TFA; gradient elution from 0% B to 90% B over 90 min) to provide N-(3-hydroxypropyl)-N-(4-(3-phenylisoxazol-5-yl)thiazol-2-yl)thiophene-2-carboxamide (565 mg) as an off-white solid.

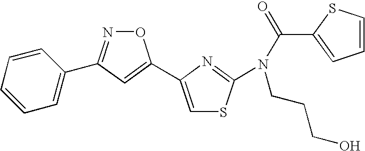

A mixture of the alcohol prepared above (230 mg, 0.56 mmol) and N,N-diisopropylethylamine (293 [L, 1.68 mmol) was dissolved in dichloromethane (25 mL) and cooled to 0° C., followed by the addition of methanesulfonyl chloride (130 μL, 1.68 mmol). The temperature of the reaction mixture was raised to ambient temperature and stirring was continuing for another 2 h. The reaction mixture was washed with water (30 mL×2) and evaporated with toluene (20 mL×2). The resulting oil was used crude in the next step without additional purification.

In a glove box under nitrogen, a mixture of D,L-proline amide hydrochloride (3 mg, 0.02 mmol) and N,N-diisopropylethylamine (7 μL, 0.04 mmol) was dissolved in NMP (200 μL) followed by the addition of the solution of the crude mesylate prepared above (10 mg, 0.2 mmol) in NMP (100 μL). The reaction mixture was maintained at room temperature overnight, and the resulting solution was purified by HPLC (Phenomenex Synergi 4 μm Max-RP column (10 mm×50 mm); flow rate=6 mL/min; injection volume=100 μL; mobile phase A: 100% water, 0.1% trifluoroacetic acid (TFA); mobile phase B: 100% acetonitrile, 0.1% trifluoroacetic acid (TFA); gradient elution from 5% B to 100% B over 6 min) to provide 1-(3-(N-(4-(3-phenylisoxazol-5-yl)thiazol-2-yl)thiophene-2-carboxamido)propyl)pyrrolidine-2-carboxamide 102 (0.4 mg) as a thin film. LC/MS (ESI) m/z 508.3 [M+H]. HPLC retention time (Method B)=2.80 min.

### Example 4

### Characterization of Compounds

The following analytical HPLC and MS conditions were used for characterizing chemical entities of the present disclosure. MS ions were detected using a Perkin-Elmer Sciex API-150 MCA atmospheric pressure chemical ionization, single quadrupole mass spectrometer interfaced to an Agilent HP 1100 HPLC system.

Method A: Symmetry C8(2) analytical column (4.6 mm×100 mm); flow rate=2.0 mL/min; injection volume=30 μL; mobile phase A: 100% water, 0.1% TFA; mobile phase, B: 100% acetonitrile, 0.1% TFA; gradient elution from 5% B to 95% B over 10.0 min, with a stay at 95% B for 4.3 min, then return to 5% B over 0.01 min and then equilibration at 5% B over 1.67 min.

Method B: Phenomenex Chromolith SpeedRod RP-18e C18 analytical column (4.6 mm×50 mm); flow rate=1.5 mL/min; injection volume=15-20 μL; mobile phase A: 100% water, 0.1% trifluoroacetic acid (TFA); mobile phase B: 100% acetonitrile, 0.1% trifluoroacetic acid (TFA); gradient elution from 5% B to 100% B over 4.2 min, with a stay at 100% B for 1 min, then equilibration to 5% B over 0.8 min.

### Example 5

Compounds listed in Tables 1 and 2 were prepared by the general procedures as exemplified in the examples, utilizing the appropriate starting materials.

TABLE 1

| | | Synthesis | LC/MS m/z | HPLC retention | HPLC |
|---|---|---|---|---|---|
| No. | ChemDraw 8.0 Name | Method | [M + H] | time (min) | Method |
| 103 | (S)-1-(3-(N-(4-(3-phenylisoxazol-5- | Example 1 | 538.1 | 5.22 | A |
| | yl)thiazol-2-yl)thiophene-2- | | | | |
| | carboxamido)propyl)-N- | | | | |
| | hydroxypiperidine-2-carboxamide | | | | |
| 104 | 1-(3-(N-(4-(3-phenylisoxazol-5- | Example 2 | 522.3 | 2.81 | B |
| | yl)thiazol-2-yl)thiophene-2- | | | | |
| | carboxamido)propyl)piperidine-2- | | | | |
| | carboxamide | | | | |
| 105 | (S)-1-(3-(N-(4-(3-phenylisoxazol-5- | Example 1 | 566.3 | 5.17 | A |
| | yl)thiazol-2-yl)thiophene-2- | | | | |
| | carboxamido)propyl)-N-(2- | | | | |
| | hydroxyethyl)piperidine-2- | | | | |
| | carboxamide | | | | |
| 106 | (S)-1-(3-(N-(4-(3-phenylisoxazol-5- | Example 1 | 536.0 | 4.99 | A |
| | yl)thiazol-2-yl)thiophene-2- | | | | |
| | carboxamido)propyl)-N- | | | | |
| | methylpiperidine-2-carboxamide | | | | |
| 107 | (S)-1-(3-(N-(4-(3-phenylisoxazol-5- | Example 1 | 508.2 | 10.1 | A |
| | yl)thiazol-2-yl)thiophene-2- | | | | |
| | carboxamido)propyl)pyrrolidine-2- | | | | |
| | carboxamide | | | | |
| 108 | (S)-1-(3-(N-(4-(3-phenylisoxazol-5- | Example 1 | 550.1 | 5.12 | A |
| | yl)thiazol-2-yl)thiophene-2- | | | | |
| | carboxamido)propyl)-N,N- | | | | |
| | dimethylpiperidine-2-carboxamide | | | | |
| 109 | (S)-1-(3-(N-(4-(3-phenylisoxazol-5- | Example 2 | 522.3 | 2.94 | B |
| | yl)thiazol-2-yl)thiophene-2- | | | | |
| | carboxamido)propyl)-N- | | | | |
| | methylpyrrolidine-2-carboxamide | | | | |
| 110 | (S)-1-(3-(N-(4-(3-phenylisoxazol-5- | Example 1 | 594.2 | 5.40 | A |
| | yl)thiazol-2-yl)thiophene-2- | | | | |
| | carboxamido)propyl)-N-tert- | | | | |
| | butoxypiperidine-2-carboxamide | | | | |

TABLE 2

| No. | ChemDraw 8.0 Name | Structure |
|---|---|---|
| 103 | (S)-1-(3-(N-(4-(3-phenylisoxazol-5-yl)thiazol-2-yl)thiophene-2-carboxamido)propyl)-N-hydroxypiperidine-2-carboxamide | 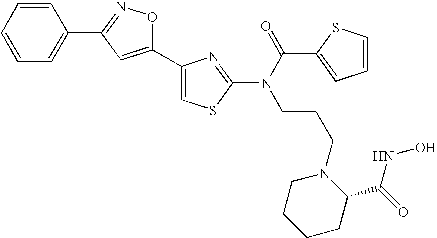 |
| 104 | 1-(3-(N-(4-(3-phenylisoxazol-5-yl)thiazol-2-yl)thiophene-2-carboxamido)propyl)piperidine-2-carboxamide | 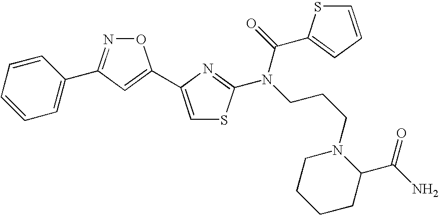 |
| 105 | (S)-1-(3-(N-(4-(3-phenylisoxazol-5-yl)thiazol-2-yl)thiophene-2-carboxamido)propyl)-N-(2-hydroxyethyl)piperidine-2-carboxamide | 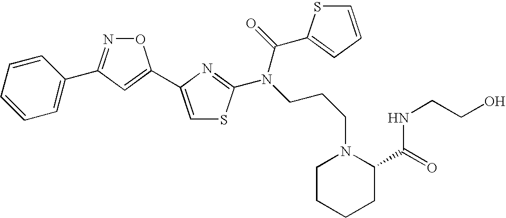 |
| 106 | (S)-1-(3-(N-(4-(3-phenylisoxazol-5-yl)thiazol-2-yl)thiophene-2-carboxamido)propyl)-N-methylpiperidine-2-carboxamide | 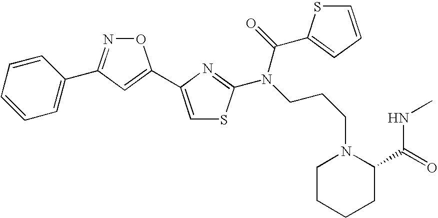 |
| 107 | (S)-1-(3-(N-(4-(3-phenylisoxazol-5-yl)thiazol-2-yl)thiophene-2-carboxamido)propyl)pyrrolidine-2-carboxamide | 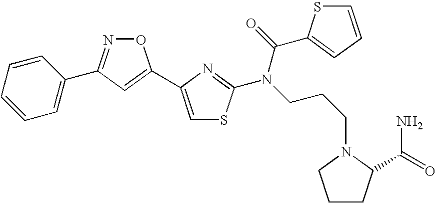 |
| 108 | (S)-1-(3-(N-(4-(3-phenylisoxazol-5-yl)thiazol-2-yl)thiophene-2-carboxamido)propyl)-N,N-dimethylpiperidine-2-carboxamide | 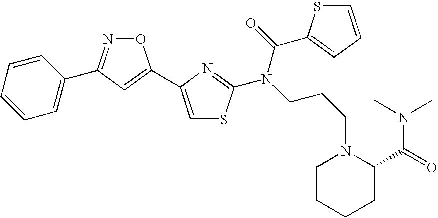 |
| 109 | (S)-1-(3-(N-(4-(3-phenylisoxazol-5-yl)thiazol-2-yl)thiophene-2-carboxamido)propyl)-N-methylpyrrolidine-2-carboxamide | 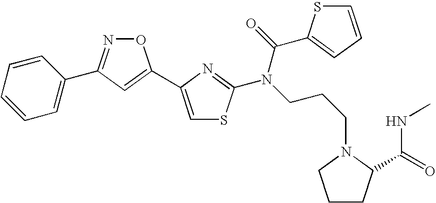 |
| 110 | (S)-1-(3-(N-(4-(3-phenylisoxazol-5-yl)thiazol-2-yl)thiophene-2-carboxamido)propyl)-N-tert-butoxypiperidine-2-carboxamide | 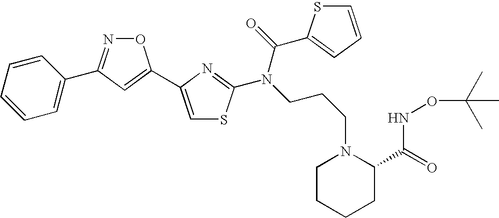 |

### Example 6

### AKT-1 Kinase Assay

The activity of the compounds described in the present invention may be determined by the following kinase assay, which measures the phosphorylation of a fluorescently-labeled peptide by full-length human recombinant active AKT-1 by fluorescent polarization using a commercially available IMAP kit.

The assay materials are obtained from an IMAP AKT Assay Bulk Kit, product #R8059, from Molecular Devices, Sunnyvale, Calif. The kit materials include an IMAP Reaction Buffer (5×). The diluted 1× IMAP Reaction Buffer contains 10 mM Tris-HCl, pH 7.2, 10 mM MgCl₂, 0.1% BSA, 0.05% NaN₃. DTT is routinely added to a final concentration of 1 mM immediately prior to use. Also included is IMAP Binding Buffer (5×), and IMAP Binding Reagent. The Binding Solution is prepared as a 1:400 dilution of IMAP Binding Reagent into 1× IMAP Binding Buffer.

The fluorescein-labeled AKT Substrate (Crosstide) has the sequence (Fl)-GRPRTSSFAEG. A stock solution of 20 μM is made up in 1× IMAP Reaction Buffer.

The plates include a Costar 3657 (382-well made of polypropylene and having a white, v-bottom) that is used for compound dilution and for preparing the compound-ATP mixture. The assay plate is a Packard ProxyPlate™-384 F.

The AKT-1 is prepared from full-length, human recombinant AKT-1 that is activated with PDK1 and MAP kinase 2.

To perform the assay, stock solutions of compounds at 10 mM in DMSO are prepared. The stock solutions and the control compound are serially diluted 1:2 nine times into DMSO (10 μL of compound+10 μL of DMSO) to give 50× dilution series over the desired dosing range. Next, 2.1-μL aliquots of the compounds in DMSO are transferred to a Costar 3657 plate containing 50 μL of 10.4 μM ATP in 1× IMAP Reaction Buffer containing 1 mM DTT. After thorough mixing, 2.5-μL aliquots are transferred to a ProxyPlate™-384 F plate.

The assay is initiated by the addition of 2.5-μL aliquots of a solution containing 200 mM of fluorescently-labeled peptide substrate and 4 nM AKT-1. The plate is centrifuged for 1 minute at 1000 g and incubated for 60 minute at ambient temperature. The reaction is then quenched by the addition of 15 μL of Binding Solution, centrifuged again and incubated for an additional 30 minutes at ambient temperature prior to reading on a Victor 1420 Multilabel HTS Counter configured to measure fluorescence polarization.

Other embodiments of the present disclosure will be apparent to those skilled in the art from consideration of the specification and practice of the present disclosure disclosed herein. It is intended that the specification and examples be considered as exemplary only, with a true scope and spirit of the present disclosure being indicated by the following claims.

What is claimed is:

1. A compound of Formula I: 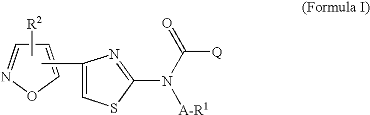
and pharmaceutically acceptable salts, chelates, non-covalent complexes, and mixtures thereof, wherein
R¹ is a 5- to 7-membered cycloheteroalkyl ring which optionally includes 1 or 2 additional heteroatoms chosen from O, S, and N in the ring and where the ring is further substituted with a group R³;
R² is chosen from phenyl and substituted phenyl;
Q is chosen from thienyl and substituted thienyl;
A is chosen from 1,3-propylene and 1,4-butylene; and
R³ is —C(O)NR⁴R⁵ wherein R⁴ and R⁵ are independently chosen from hydrogen, hydroxy, hydroxyethyl, lower alkyl, and lower alkoxy.

2. The compound of claim 1 having the structure:

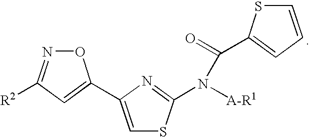

3. The compound of claim 1 having the structure:

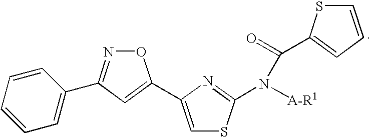

4. The compound of claim 1 wherein R¹ is chosen from pyrrolidine, piperidine, azepane, piperazine, and morpholine, each of which is further substituted with a group R³.

5. The compound of claim 4 wherein R¹ is chosen from piperidine further substituted with a group R³.

6. The compound of claim 1 wherein R⁴ is hydrogen.

7. The compound of claim 1 wherein R⁵ is chosen from hydrogen, hydroxy, hydroxyethyl, and lower alkyl.

8. The compound of claim 7 wherein R⁵ is chosen from hydrogen, hydroxy, hydroxyethyl, and methyl.

9. The compound of claim 1 wherein R² is phenyl.

10. The compound of claim 1 wherein Q is thienyl.

11. The compound of claim 1 wherein A is 1,3-propylene.

12. The compound of claim 1 wherein the compound is an inhibitor of at least one ATP-utilizing enzyme.

13. The compound of claim 12 wherein the at least one ATP-utilizing enzyme is chosen from a human protein kinase.

14. The compound of claim 13 wherein the human protein kinase is chosen from AKT1 and PIM1 kinase.

15. The compound of claim 13 wherein the human protein kinase is AKT1.

16. At least one chemical entity of claim 1 wherein the compound of Formula I is chosen from
(S)-1-(3-(N-(4-(3-phenylisoxazol-5-yl)thiazol-2-yl)thiophene-2-carboxamido)propyl)piperidine-2-carboxamide;
(S)-1-(3-(N-(4-(3-phenylisoxazol--yl)thiazol-2-yl)thiophene-2-carboxamido)propyl)-N-hydroxypiperidine-2-carboxamide;
1-(3-(N-(4-(3-phenylisoxazol-5-yl)thiazol-2-yl)thiophene-2-carboxamido)propyl)piperidine-2-carboxamide;
(S)-1-(3-(N-(4-(3-phenylisoxazol--yl)thiazol-2-yl)thiophene-2-carboxamido)propyl)-N-(2-hydroxyethyl)piperidine-2-carboxamide;
(S)-1-(3-(N-(4-(3-phenylisoxazol-5-yl)thiazol-2-yl)thiophene-2-carboxamido)propyl)-N-methylpiperidine-2-carboxamide;
(S)-1-(3-(N-(4-(3-phenylisoxazol-5-yl)thiazol-2-yl)thiophene-2-carboxamido)propyl)pyrrolidine-2-carboxamide;
(S)-1-(3-(N-(4-(3-phenylisoxazol-5-yl)thiazol-2-yl)thiophene-2-carboxamido)propyl)-N,N-dimethylpiperidine-2-carboxamide;
1-(3-(N-(4-(3-phenylisoxazol-5-yl)thiazol-2-yl)thiophene-2-carboxamido)propyl)pyrrolidine-2-carboxamide;
(S)-1-(3-(N-(4-(3-phenylisoxazol-5-yl)thiazol-2-yl)thiophene-2-carboxamido)propyl)-N-methylpyrrolidine-2-carboxamide; and
(S)-1-(3-(N-(4-(3-phenylisoxazol-5-yl)thiazol-2-yl)thiophene-2-carboxamido)propyl)-N-tert-butoxypiperidine-2-carboxamide.

17. A pharmaceutical composition comprising at least one pharmaceutically acceptable vehicle, and a therapeutically effective amount of at least one compound of claim 1.

18. The pharmaceutical composition of claim 17 further comprising at least one additional therapeutic agent appropriate for effecting combination therapy.

19. The pharmaceutical composition of claim 18 wherein said at least one additional therapeutic agent appropriate for effecting combination therapy is chosen from estrogen receptor modulators, cytostatic/cytotoxic agents, anti-proliferative agents, cell cycle checkpoint inhibitors, angiogenesis inhibitors, monoclonal antibody targeted therapeutic agents, tyrosine kinase inhibitors, serine-threonine kinase inhibitors, histone deacetylase inhibitors, heat shock protein inhibitors, and farnesyl transferase inhibitors.

20. A method of treating cancer in a patient in need of such treatment comprising administering to the patient a therapeutically effective amount of at least one compound of claim 1, wherein the cancer is glioblastoma, ovarian cancer, breast cancer, endometrial carcinoma, hepatocellular carcinoma, melanoma, colorectal cancer, colon cancer, digestive tract, lung cancer, thyroid, lymphoid, prostate cancer, advanced tumors, hairy cell leukemia, melanoma, chronic myelogenous leukemia, advanced head and neck, squamous cell cancer, metastatic renal cell, non-Hodgkin's lymphoma, metastatic breast, breast adenocarcinoma, advanced melanoma, pancreatic, gastric, non-small cell lung, small cell lung, renal cell carcinoma, multiple myeloma, metastatic prostate, malignant glioma, renal cancer, lymphoma refractory metastatic disease, refractory multiple myeloma, cervical cancer, Kaposi's sarcoma, recurrent anaplastic glioma, or metastatic colon cancer.

21. The method of claim 20, further comprising administering at least one additional therapeutic agent appropriate for effecting combination therapy.

22. The method of claim 21 wherein said at least one additional therapeutic agent appropriate for effecting combination therapy is chosen from estrogen receptor modulators, cytostatic/cytotoxic agents, anti-proliferative agents, cell cycle checkpoint inhibitors, angiogenesis inhibitors, monoclonal antibody targeted therapeutic agents, tyrosine kinase inhibitors, serine-threonine kinase inhibitors, histone deacetylase inhibitors, heat shock protein inhibitors, and farnesyl transferase inhibitors.

23. A method of inhibiting at least one ATP-utilizing enzyme in a subject comprising administering to the subject at least one compound of claim 1.

24. The method of claim 23 wherein the at least one ATP-utilizing enzyme is chosen from a human protein kinase.

25. The method of claim 24 wherein the human protein kinase is chosen from AKT1 and PIMN kinase.

26. The method of claim 24 wherein the human protein kinase is AKT1.

27. A packaged pharmaceutical formulation comprising a pharmaceutical composition of claim 17 and instructions for using the composition to treat a mammal.

28. The packaged pharmaceutical formulation of claim 27 wherein the instructions are for using the pharmaceutical composition to treat a patient suffering from a disease responsive to inhibition at least one ATP-utilizing enzyme.

29. The use of at least one compound of claim 1 in the manufacture of a medicament for treating cancer.

30. The use of claim 29 wherein cancer is glioblastoma, ovarian cancer, breast cancer, endometrial carcinoma, hepatocellular carcinoma, melanoma, colorectal cancer, colon cancer, digestive tract, lung cancer, thyroid, lymphoid, prostate cancer, advanced tumors, hairy cell leukemia, melanoma, chronic myelogenous leukemia, advanced head and neck, squamous cell cancer, metastatic renal cell, non-Hodgkin's lymphoma, metastatic breast, breast adenocarcinoma, advanced melanoma, pancreatic, gastric, non-small cell lung, small cell lung, renal cell carcinoma, multiple myeloma, metastatic prostate, malignant glioma, renal cancer, lymphoma refractory metastatic disease, refractory multiple myeloma, cervical cancer, Kaposi's sarcoma, recurrent anaplastic glioma, or metastatic colon cancer.
